(19) United States
(12) Patent Application Publication
(10) Pub. No.: US 2006/0024232 A1
(43) Pub. Date: Feb. 2, 2006

(54) Imaging and therapeutic agents targeting proteins expressed on endothelial cell surface

(75) Inventors: Jan E. Schnitzer, Encinitas, CA (US); Philip Oh, San Diego, CA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Sidney Kimmel Cancer Center, San Diego, CA (US)

(21) Appl. No.: 11/143,919

(22) Filed: Jun. 2, 2005

Related U.S. Application Data
(60) Provisional application No. 60/576,192, filed on Jun. 2, 2004.

(51) Int. Cl. A61K 51/00; A61K 39/395; A61K 48/00

(52) U.S. Cl. 424/1.49; 424/143.1; 514/44; 424/9.34; 424/9.6

(57) ABSTRACT

Methods of delivering an agent in a tissue-specific manner, by targeting proteins expressed on endothelial cell surface, are described. The methods can be used for detecting, imaging and/or treating neoplasia, angiogenesis or neovasculature, as well as for diagnostics and methods of assessing treatment efficacy.

### RELATED APPLICATION

This application claims the benefit of U.S. Provisional Application No. 60/576,192, filed Jun. 2, 2004. The entire teachings of the above application are incorporated herein by reference.

### GOVERNMENT SUPPORT

The invention was supported, in whole or in part, by grants HL58216, HL52766, CA95893, CA83989 and CA97528 from the National Institutes of Health. The Government has certain rights in the invention.

### BACKGROUND OF THE INVENTION

New targets are needed for detecting disease through molecular imaging (Massoud, T. F. & Gambhir, S. S., Genes Dev 17, 545-80 (2003); Herschman, H. R., Science 302, 605-8 (2003); Rudin, M. & Weissleder, R., Nat Rev Drug Discov 2, 123-31 (2003); Weissleder, R. Nat Rev Cancer 2, 11-8 (2002)) and for treating disease through directed delivery in vivo (Drews, J., Science 287, 1960-4 (2000); Lindsay, M. A., Nat Rev Drug Discov 2, 831-8 (2003); Workman, P., Curr Cancer Drug Targets 1, 33-47 (2001); Anzick, S. L. & Trent, J. M. Oncology (Huntingt) 16, 7-13 (2002); Cavenee, W. K., Carcinogenesis 23, 683-6 (2002)). Genome completion identifies a target pool of 40,000 genes which may translate into a million possible protein targets (Huber, L. A., Nat Rev Mol Cell Biol 4, 74-80 (2003)). Genomic and proteomic analysis of normal and diseased tissues have yielded thousands of genes and gene products for diagnostic and tissue assignment as well as potential therapeutic targeting (Drews, J., Science 287, 1960-4 (2000); Lindsay, M. A., Nat Rev Drug Discov 2, 831-8 (2003); Workman, P., Curr Cancer Drug Targets 1, 33-47 (2001); Anzick, S. L. & Trent, J. M., Oncology (Huntingt) 16, 7-13 (2002); Huber, L. A., Nat Rev Mol Cell Biol 4, 74-80 (2003); Perou, C. M. et al., Nature 406, 747-52 (2000)). Yet the sheer number of candidates can overwhelm the required in vivo validation process, leading some to question the ultimate impact of these approaches on speeding up drug discovery (Drews, J., Science 287, 1960-4 (2000); Lindsay, M. A., Nat Rev Drug Discov 2, 831-8 (2003); Workman, P., Curr Cancer Drug Targets 1, 33-47 (2001); Huber, L. A., Nat Rev Mol Cell Biol 4, 74-80 (2003)). Reducing tissue data complexity to a manageable subset of candidates most relevant to targeting, imaging, and treating disease is clearly desired but requires new discovery and validation strategies that effectively focus the power of global identification technologies.

Selectively targeting a single organ or diseased tissue such as solid tumors in vivo remains a desirable yet elusive goal of molecular medicine that could enable more effective imaging as well as drug and gene therapies for many acquired and genetic diseases (Massoud, T. F. & Gambhir, S. S. Genes Dev 17, 545-80 (2003); Herschman, H. R., Science 302, 605-8 (2003); Weissleder, R., Nat Rev Cancer 2, 11-8 (2002); Lindsay, M. A., Nat Rev Drug Discov 2, 831-8 (2003); Huber, L. A., Nat Rev Mol Cell Biol 4, 74-80 (2003)). Most tissue- and disease-associated proteins are expressed by cells inside tissue compartments not readily accessible to intravenously injected biological agents such as antibodies. This inaccessibility hinders many site-directed therapies (Drews, J., Science 287, 1960-4 (2000); Lindsay, M. A., Nat Rev Drug Discov 2, 831-8 (2003); Workman, P., Curr Cancer Drug Targets 1, 33-47 (2001); Jain, R. K., Nat. Med. 4, 655-7 (1998); Dvorak, H. F., et al., Cancer Cells 3, 77-85 (1991)) and imaging agents (Massoud, T. F. & Gambhir, S. S., Genes Dev 17, 545-80 (2003); Herschman, H. R., Science 302, 605-8 (2003); Rudin, M. & Weissleder, R., Nat Rev Drug Discov 2, 123-31 (2003); Weissleder, R. Nat Rev Cancer 2, 11-8 (2002)). For example, multiple barriers to solid tumor delivery prevent effective immunotherapy in vivo, despite efficacy and specificity in vitro (Jain, R. K., Nat. Med. 4, 655-7 (1998); Dvorak, H. F., et al., Cancer Cells 3, 77-85 (1991); von Mehren, M., et al., Annu Rev Med 54, 343-69 (2003); Farah, R. A., et al., Crit Rev Eukaryot Gene Expr 8, 321-56 (1998); Carver, L. A. & Schnitzer, J. E., Nat Rev Cancer 3, 571-81 (2003); Schnitzer, J. E., N Engl J Med 339, 472-4 (1998)). Conversely, the universal access of chemotherapeutics dilutes efficacy to require increased dosages leading to unwanted systemic side effects. Thus, new approaches are required that cut through the cumbersome overabundance of molecular information to permit rapid discovery and validation of accessible tissue-specific targets that can direct molecular imaging and pharmacodelivery in vivo.

Vascular endothelial cells form a barrier in vivo that can greatly limit the ability of many drugs, gene vectors, and imaging agents circulating in the blood to reach their intended target cells residing within a single tissue. This restricted accessibility can prevent therapeutic efficacy in vivo and increase therapeutic side effects. Vascular targeting is a new drug and gene delivery strategy that targets the luminal endothelial cell surface and its caveolae which are directly exposed and thus inherently accessible to agents circulating in the blood (McIntosh, D. P., et al., Proc Natl Acad Sci USA 99, 1996-2001 (2002); Carver, L. A. & Schnitzer, J. E., Nat Rev Cancer 3, 571-581 (2003)). Agents such as peptides and antibodies to endothelial cell surface proteins show promise for directing tissue-specific pharmacodelivery to the vasculature in vivo (McIntosh, D. P., et al., Proc Natl Acad Sci USA 99, 1996-2001 (2002); Pasqualini, R. & Ruoslahti, E., Nature 380, 364-366 (1996); Muzykantov, V. R., et al., Proc Natl Acad Sci USA 93, 5213-5218 (1996); Muzykantov, V. R. et al., Proc Natl Acad Sci USA 96, 2379-2384. (1999)) but greater molecular information and more candidate targets expressed in vivo are needed to understand and define the potential of vascular targeting.

The endothelium exists as an attenuated cell monolayer lining all blood vessels, and forming a physiologically vital interface between the circulating blood and the underlying cells inside the tissue. It plays a significant role controlling the passage of blood molecules and cells into the tissue and in many other normal physiological functions including vasoregulation, coagulation, and inflammation as well as tissue nutrition, growth, survival, repair and overall organ homeostasis and function (Schnitzer, J. E., Trends in Cardiovasc. Med. 3, 124-130 (1993)). Disruption of the vascular endothelium and its normal barrier function can lead rapidly to tissue edema, hypoxia, pathology, and even organ death (Fajardo, L. F., Am. J. Clin. Pathol. 92, 241-250 (1989); Jaffe, E. A., Hum. Pathol. 18, 234-239 (1987)).

Although the microenvironment of the tissue surrounding the blood vessels appears clearly to influence greatly the phenotype of the endothelial cells (Madri, J. A. & Williams, S. K., J. Cell Biol. 97, 153-165 (1983); Goerdt, S. et al., Exp Cell Biol 57, 185-192 (1989); Gumkowski, F. et al., Blood Vessels 24, 11-23 (1987); Hagemeier, H. H., et al., Int J Cancer 38, 481-488. (1986); Aird, W. C. et al., J Cell Biol 138, 1117-1124 (1997); Janzer, R. C. & Raff, M. C. Nature 325, 253-257 (1987); Stewart, P. A. & Wiley, M. J., Develop Biol 84, 183-192 (1981)), currently there is very little molecular information about vascular endothelium as it exists natively in the tissue. This is in large part because of technical limitations in performing large-scale molecular profiling on a cell-type that comprises such a small percentage of the total cells in the tissue. Past approaches have relied primarily on genomic or antibody-based analysis of endothelial cells isolated from the tissue by enzymatic digestion to disassemble the tissue and release single cells for sorting using endothelial cell markers (Auerbach, R., et al.,. Microvasc Res 29, 401-411 (1985); St Croix, B. et al., Science 289, 1197-1202 (2000); Plendl, J., et al.,. Anat Histol Embryol 21, 256-262 (1992)). Over the last three decades, the study of isolated and even cultured endothelial cells has yielded much functional and molecular information; however, both the significant perturbation of the tissue and the growth in culture contribute to morphologically obvious phenotypic drift that can translate rapidly into loss of native function and protein expression (Madri, J. A. & Williams, S. K., J. Cell Biol. 97, 153-165 (1983); Schnitzer, J. E. in Capillary Permeation, Cellular Transport and Reaction Kinetics. (ed. J. H. Linehan) 31-69 (Oxford Press, London; 1997). The reported ability of specific cells and select peptides displayed on bacteriophage to home to specific tissues of the body after intraveous injection also provides indirect evidence supporting the molecular heterogeneity of endothelial cell surface in different organ (Pasqualini, R. & Ruoslahti, E., Nature 380, 364-366 (1996); Plendl, J., et al., Anat Histol Embryol 21, 256-262 (1992); Rajotte, D. et al., J Clin Invest 102, 430-437 (1998)) but have not yet facilitated mapping of endothelial cell surface proteins in vivo. The degree to which endothelial cell expression is modulated within different normal and diseased tissues remains unclear.

### SUMMARY OF THE INVENTION

The present invention pertains to methods of delivering an agent to, into and/or across vascular endothelium in a neoplasm-specific manner. In the methods of the invention, the agent is delivered by contacting the luminal surface of vasculature, or caveolae of the vasculature, with an agent that specifically binds to a targeted protein expressed on endothelial cell surface. In preferred embodiments, the targeted protein is VEGF receptor 1, VEGF receptor 2, Tie-2, aminopeptidase N, endoglin, C-CAM-1, neuropilin-1, AnnA1, AnnA8, EphA5, EphA7, myeloperoxidase, neucleolin, transferrin receptor, or vitamin D binding receptor.

In certain embodiments of the invention, the methods can be used for treating neoplasia in an individual, by administering to the individual a therapeutic targeting agent that binds to a targeted protein expressed on endothelial cell surface. The therapeutic targeting agent can be an antibody to the targeted protein expressed on endothelial cell surface; alternatively, the therapeutic targeting agent can be a binding partner of a targeted protein expressed on endothelial cell surface. In addition, the therapeutic targeting agent can also be an agent having an active agent component and a targeting agent component, in which the targeting agent component is: an agent that specifically binds to a targeted protein expressed on endothelial cell surface (e.g., an antibody to the targeted protein expressed on endothelial cell surface); or a specific binding partner of the targeted protein expressed on endothelial cell surface. In these embodiments, the active agent component can be, for example, a radionuclide; a chemotherapeutic agent; an immune stimulatory agent; an anti-neoplastic agent: an anti-inflammatory agent; a pro-inflammatory agent; a pro-apoptotic agent; a pro-coagulant; a toxin; an antibiotic; a hormone; an enzyme; a protein (e.g., a recombinant protein or a recombinant modified protein) a carrier protein (e.g., albumin, modified albumin); a lytic agent; a small molecule; aptamers; cells, including modified cells; vaccine-induced or other immune cells; nanoparticles (e.g., albumin-based nanoparticles); transferring; immunoglobulins; multivalent antibodies; lipids; lipoproteins; liposomes; an altered natural ligand; a gene or nucleic acid; RNA; siRNA; a viral or non-viral gene delivery vector; a prodrug; or a promolecule.

The invention also pertains to methods of assessing response to treatment with a therapeutic targeting agent, by assessing the level of the targeted protein expressed on endothelial cell surface, in a sample from the individual before treatment with the therapeutic targeting agent, and during or after treatment with the therapeutic targeting agent, and comparing the levels; a level of the targeted protein during or after treatment that is significantly lower than the level of the targeted protein before treatment, is indicative of efficacy of treatment with the therapeutic targeting agent.

The invention further pertains to methods for performing physical imaging of an individual, using an imaging agent that includes a targeting agent component (as described above) and an imaging agent component. The imaging agent component can be, for example, a radioactive agent, radioisotope or radiopharmaceutical; a contrast agent; a magnetic agent or a paramagnetic agent; liposomes; nanoparticles; ultrasound agents; a gene vector or virus inducing a detecting agent; an enzyme; a prosthetic group; a fluorescent material; a luminescent material; or a bioluminescent material. Upon administration, the targeted imaging agents can be visualized noninvasively by conventional external detection means (designed for the imaging agent), to detect the preferential or specific accumulation in the neoplasm. In addition, the invention pertains to methods of delivering such imaging agents in vivo in a neoplasm-specific manner, and then assessing a biopsy sample for the presence of the imaging agent; the methods also pertain to delivering imaging agents in a neoplasm-specific manner to a tissue sample. In addition, the invention pertains to methods of delivering such imaging agents in a neoplasm-specific manner to a tissue (e.g., tumor) sample. The methods additionally pertain to methods assessing an individual for the presence or absence of neoplasia, administering to the individual an agent of interest that comprises an imaging agent component and a targeting agent component, as described above, and assessing the individual for the presence or absence of a concentration of the agent of interest, wherein the presence of a concentration of the agent of interest is indicative of the presence of neoplasia.

The present invention additionally pertains to methods of delivering agents to, into and/or across vascular endothelium in an neovasculature-specific manner. In the methods of the invention, the agent is delivered by contacting the luminal surface of vasculature, or caveolae of vasculature, with an agent that specifically binds to a targeted protein expressed on endothelial cell surface. In preferred embodiments, the targeted protein is VEGF receptor 1, VEGF receptor 2, Tie-2, aminopeptidase N, endoglin, C-CAM-1, neuropilin-1, AnnA1, AnnA8, EphA5, EphA7, myeloperoxidase, neucleolin, transferrin receptor, or vitamin D binding receptor.

In certain embodiments of the invention, the methods can be used for treating neovasculature (e.g., angiogenesis, the development of undesirable neovasculature) in an individual, by administering to the individual a therapeutic targeting agent that binds to a targeted protein expressed on endothelial cell surface. The therapeutic targeting agent can be an antibody to the targeted protein expressed on endothelial cell surface; alternatively, the therapeutic targeting agent can be a binding partner of a targeted protein expressed on endothelial cell surface. In addition, the therapeutic targeting agent can also be an agent having an active agent component and a targeting agent component, in which the targeting agent component is: an agent that specifically binds to a targeted protein expressed on endothelial cell surface (e.g., an antibody to the targeted protein expressed on endothelial cell surface); or a specific binding partner of the targeted protein expressed on endothelial cell surface. In a preferred embodiment, the targeting agent component is an agent that specifically binds to a targeted protein expressed during angiogenesis or during the development of neovasculature. In these embodiments, the active agent component can be, for example, a radionuclide; a chemotherapeutic agent; an immune stimulatory agent; an anti-neoplastic agent: an anti-inflammatory agent; a pro-inflammatory agent; a pro-apoptotic agent; a pro-coagulant; toxin; an antibiotic; a hormone; an enzyme; a protein (e.g., a recombinant protein or a recombinant modified protein) a carrier protein (e.g., albumin, modified albumin); a lytic agent; a small molecule; aptamers; cells, including modified cells; vaccine-induced or other immune cells; nanoparticles (e.g., albumin-based nanoparticles); transferring; immunoglobulins; multivalent antibodies; lipids; lipoproteins; liposomes; an altered natural ligand; a gene or nucleic acid; RNA or siRNA; a viral or non-viral gene delivery vector; a prodrug; or a promolecule.

In certain other embodiments of the invention, the methods can be used for enhancing or increasing neovasculature in an individual, by administering to the individual a therapeutic neovasculature targeting agent.

In addition, the invention pertains to methods of delivering such imaging agents in vivo in an neovasculature-specific manner, and then assessing a biopsy sample for the presence of the imaging agent; the methods also pertain to delivering imaging agents in an neovasculature-specific manner to a tissue sample. The methods additionally pertain to methods of assessing an individual for the presence or absence of neovasculature, administering to the individual an agent of interest that comprises an imaging agent component and a targeting agent component, as described above, and assessing the individual for the presence or absence of a concentration of the agent of interest, wherein the presence of a concentration of the agent of interest is indicative of the presence of neovasculature.

The methods of the invention provide an easy method that permits imaging of certain tissues or groups of tissues, and also permits specific delivery to, penetration into, imaging of, and destruction of neoplasms or neovasculature in vivo. In addition, it allows use of the described agents for manufacture of medicaments for use in delivery to, treatment of, and/or imaging of neoplasms or neovasculature.

### DETAILED DESCRIPTION OF THE INVENTION

A description of preferred embodiments of the invention follows.

An analytical paradigm was developed that reduced the complexity of normal and diseased tissue to focus on a small subset of proteins induced at the blood-tissue interface. Subcellular tissue fractionation, subtractive proteomics, in silico bioinformatics, expression profiling, and molecular imaging were integrated to map tissue-modulation of luminal endothelial cell surface protein expression in vivo and to discover and rapidly validate intravenous-accessible targets permitting specific immunotargeting of neoplasms in vivo.

The analytical paradigm confirmed the expression of several proteins as being associated with tumors, including seven proteins already implicated in tumor angiogenesis (VEGF receptors 1 and 2, Tie-2, aminopeptidase N, endoglin, C-CAM-1, and neuropilin-1). These proteins revealed a stronger signal in lung tumor, although each protein was also readily detected in normal lung. In addition, eight tumor-induced vascular proteins were identified: AnnA1, AnnA8, EphA5, EphA7, myeloperoxidase, nucleolin, transferrin receptor, and vitamin D binding receptor. Of these, AnnA1 and vitamin D binding protein were detected in tumor but not in normal lung; seven of the eight were expressed more in tumor than in normal lung. As a result of this discovery, methods are now available to deliver agents to, into and/or across vascular endothelium in a neoplasm-specific manner, using an agent that specifically binds to a targeted protein. In certain embodiments of the invention, the methods deliver a therapeutic agent to, into and/or across vascular endothelium in a neoplasm-specific manner. It is believed that delivery to, into, and/or across luminal surface of the vascular endothelium of a neoplasm can transport into/across the endothelial cell or another barrier and ultimately allow delivery of agents into the interstitium of a neoplasm, allowing an agent to be delivered to all areas of a neoplasm (including endothelial, stromal, and other parts of a neoplasm). These methods can be used to treat neoplasias in an individual. In other embodiments of the invention, the methods deliver an imaging agent to, into and/or across vascular endothelium in a neoplasm-specific manner. Also available are in vivo and in vitro diagnostics, utilizing an agent that specifically binds to a targeted protein, as well as methods to assess treatment efficacy as well as to assess prognosis of disease. In addition, methods are now available to deliver agents to, into and/or across vascular endothelium in an neovasculature-specific manner, using an agent that specifically binds to a targeted protein. It is believed that delivery to, into, and/or across vascular endothelium can allow an agent to be delivered to areas comprising neovasculature.

Vascular Endothelium and Tissue and Tumor Accessibility

Plasmalemmal vesicles called caveolae are abundant on the endothelial cell surface, function in selective endocytosis and transcytosis of nutrients, and provide a means to enter endothelial cells (endocytosis) and/or to penetrate the endothelial cell barrier (transcytosis) for delivery to underlying tissue cells. Focus is now on the vascular endothelial cell surface in contact with the circulating blood, to bypass the problem of poor penetrability into tumors; this vascular endothelial cell surface provides an inherently accessible, and thus targetable, surface. Intravenously-accessible neovascular targets induced in tumors and not expressed in the endothelium of normal organs are required for this strategy to achieve its theoretical expectation.

Past work has mapped and characterized the molecular architecture and function of the cell surface and especially its caveolae in normal vascular endothelium, primarily in rat lung tissue (Schnitzer, J. E. and Oh, P. (1994) J Biol Chem 269, 6072-82; Schnitzer, J. E., et al., (1994) J Cell Biol 127, 1217-32; Schnitzer, J. E., et al., (1995) Science 269, 1435-9; Schnitzer, J. E., et al., (1996) [publisher's erratum appears in Science 1996 Nov. 15; 274(5290):1069]. Science 274, 239-42; Schnitzer, J. E., et al., (1995). J Biol Chem 270, 14399-404; Schnitzer, J. E., et al., (1995) Am J Physiol 268, H48-55; McIntosh, D. P. and Schnitzer, J. E. (1999) Am J Physiol 277, H2222-32).

Targeting endothelial caveolae via antibodies or other agents that specifically bind to proteins expressed on the cell surface of vascular endothelium, permits specific delivery to, penetration into, and imaging of tissues. Furthermore, these methods can be used to aid in targeting neoplasms in vivo, for example, for destruction or for imaging. As used herein, the term “targeted protein” refers to a protein, such as one of the proteins identified in the Examples, that is expressed on the endothelial cell surface. The targeted proteins described herein include a variety of proteins, including:
proteins that are associated with tumors, including proteins that are expressed to a greater degree in tumor tissue than in comparable normal tissue (e.g., VEGF receptor 1, VEGF receptor 2, Tie-2, aminopeptidase N, endoglin, C-CAM-1, neuropilin-1);
proteins that are tumor-induced vascular proteins expressed to a greater degree in tumor tissue than in comparable normal tissue (e.g., AnnA8, EphA5, EphA7, myeloperoxidase, nucleolin, transferrin receptor); and
proteins that are expressed primarily in tumor tissue and not in significant amounts in comparable normal tissue (e.g., AnnA1 and vitamin D binding protein).

A protein that is expressed to “a greater degree” in neoplastic tissue than in comparable normal tissue is a protein that is expressed in an amount that is greater, by a degree that is significant (e.g., equal to or greater than 2-fold, preferably equal to or greater than 3-fold, even more preferably equal to or greater than 5-fold, still more preferably equal to or greater than 10-fold, even more preferably equal to or greater than 20-fold) than the expression of that protein in a comparable normal tissue. A comparable normal tissue is a neoplasm-free tissue of the same type as the neoplasm tissue (e.g., lung tissue is a comparable normal tissue for lung neoplasm). The selection of which type of targeted protein for use in the invention will depend on the desired targeting methods. When the degree of expression is greater by a higher degree (e.g., equal to or greater than 10-fold, equal to or greater than 20-fold, or even equal to or greater than 100-fold), the expression may become functionally equivalent to expression solely in the neoplastic tissue: directed and effective delivery of agents (e.g., therapeutic agents or imaging agents as described herein) to the neoplasm tissue occurs, with minimal or no delivery to other tissues. Thus, the amount that is functionally equivalent to expression solely in the neoplastic tissue can be determined by assessing whether the goal of effective delivery of agents is met with minimal or no delivery to other tissues.

Delivery of Agents

In certain methods of the invention, an agent is delivered in a neoplasm-specific manner, utilizing an agent that specifically binds to a protein expressed on neoplasm endothelial cell surface. It is believed that delivery to, into, and/or across vascular endothelium of a neoplasm can allow delivery of agents into the interstitium of a neoplasm, allowing penetration of an agent to be delivered to all areas of a neoplasm (including, for example, endothelial, stromal, and most, if not all, other parts of a tumor). Similarly, in certain other methods of the invention, an agent is delivered in an neovasculature-specific manner, using an agent that specifically binds to a protein expressed during neovasculature. It is believed that delivery to, into, and/or across vascular endothelium can allow an agent to be delivered to areas comprising neovasculature.

In certain embodiments of the invention, the methods deliver a therapeutic agent to, into and/or across vascular endothelium in a neoplasm-specific manner. These methods can be used to treat neoplasias or other disease states in an individual. The term, “neoplasm,” as used herein refers particularly to malignant neoplasms, and includes not only to sarcomas (e.g., fibrosarcoma, myosarcoma, liposarcoma, chondrosarcoma, hemangiosarcoma, mesothelioma, leukemias, lymphomas, leiomyosarcoma, rhabdomyosarcoma), but also to carcinomas (e.g., adenocarcinoma, papillary carcinoma, cystadenocarcinoma, melanoma, renal cell carcinoma, hepatoma, choriocarcinoma, seminoma), as well as mixed neoplasms (e.g., teratomas). Thus, “neoplasm” contemplates not only solid tumors, but also so-called “soft” tumors. Furthermore, “neoplasm” contemplates not only primary neoplasms, but also metastases. In representative embodiments, neoplasms that can be targeted include brain, breast, lung, kidney, prostate, ovarian, head and neck, and liver tumors.

In other embodiments of the invention, the methods deliver an imaging agent to, into and/or across vascular endothelium in a neoplasm-specific manner. In certain other embodiments of the invention, the methods deliver a neovasculature therapeutic agent to, into and/or vascular endothelium in a neovasculature-specific manner. These methods can be used to treat undesirable neovasculature or other disease states in an individual. In other embodiments of the invention, the methods deliver an imaging agent to, into and/or across vascular endothelium in a neovasculature-specific manner. In further embodiments of the invention, the methods deliver a neovasculature therapeutic agent to, into and/or across vascular endothelium in a neovasculature-specific manner in order to enhance or increase neovasculature if desired.

An agent that “specifically binds” to a targeted protein, as the term is used herein, is an agent that preferentially or selectively binds to that targeted protein. While certain degree of non-specific interaction may occur between the agent that specifically binds and the targeted protein, nevertheless, specific binding, may be distinguished as mediated through specific recognition of the targeted protein, in whole or part.

Typically specific binding results in a much stronger association between the agent and the targeted protein than between the agent and other proteins, e.g., other vascular proteins. The affinity constant (Ka, as opposed to Kd) of the agent for its cognate is at least 10⁶ or 10⁷, usually at least 10⁸, alternatively at least 10⁹, alternatively at least 10¹⁰, or alternatively at least 10¹¹ M. It should be noted, also, that “specific” binding may be binding that is sufficiently site-specific to effectively be “specific”: for example, when the degree of binding is greater by a higher degree (e.g., equal to or greater than 10-fold, equal to or greater than 20-fold, or even equal to or greater than 100-fold), the binding may become functionally equivalent to binding solely to the targeted protein at a particular location: directed and effective binding occurs with minimal or no delivery to other tissues. Thus, the amount that is functionally equivalent to specific binding can be determined by assessing whether the goal of effective delivery of agents is met with minimal or no binding to other tissues.

In a particular embodiment, the agent that specifically binds the targeted protein is or comprises an antibody or fragment of an antibody (e.g., Fab′ fragments). Representative antibodies include commercially available antibodies (as listed in Linscott's Directory). Alternatively, the agent is or comprises another agent that specifically binds to a targeted protein (a “specific binding partner”). Representative specific binding partners include natural ligands, peptides, small molecules (e.g., inorganic small molecules, organic small molecules, derivatives of small molecules, composite small molecules); aptamers; cells, including modified cells; vaccine-induced or other immune cells; nanoparticles (e.g, lipid or non-lipid based formulations); lipids; lipoproteins; lipopeptides; lipid derivatives; liposomes; modified endogenous blood proteins used to carry chemotherapeutics; a protein (e.g., a recombinant protein or a recombinant modified protein) a carrier protein (e.g., albumin, modified albumin); a lytic agent; a small molecule; other nanoparticles (e.g., albumin-based nanoparticles); transferrins; immunoglobulins; multivalent antibodies; lipids; lipoproteins; liposomes; an altered natural ligand; a gene or nucleic acid; RNA or siRNA; a viral or non-viral gene delivery vector; a prodrug; or a promolecule.

The agent can also comprise a first component that binds to the targeted protein, as described above, and a second component, that is an active component (e.g., a therapeutic agent or imaging agent, as described in detail below). The agent can be administered by itself, or in a composition (e.g., a pharmaceutical or physiological composition) comprising the agent. It can be administered either in vivo (e.g., to an individual) or in vitro (e.g., to a tissue sample). The methods of the invention can be used not only for human individuals, but also are applicable for veterinary uses (e.g., for other mammals, including domesticated animals (e.g., horses, cattle, sheep, goats, pigs, dogs, cats) and non-domesticated animals.

The agent can be administered by itself, or in a composition (e.g., a physiological or pharmaceutical composition) comprising the agent. For example, the therapeutic targeting agent can be formulated together with a physiologically acceptable carrier or excipient to prepare a pharmaceutical composition. The carrier and composition can be sterile. The formulation should suit the mode of administration.

If desired, non-specific background and/or scavenger uptake of agents by reticulo-endothelial system (primarily liver and spleen) may be reduced by overwhelming the system by inhibition and/or competitions with various reagents, including, for example, immunoglobulins, proteins or protein fragments, starches or hydroxyethylstarches, albumins, modified albumins, or other agents. Such agents can be administered prior to, or concurrently with, the agents of the invention.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active agents. The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, etc.

Methods of introduction of these compositions include, but are not limited to, intradermal, intramuscular, intraperitoneal, intraocular, intravenous, subcutaneous, topical, oral and intranasal. Other suitable methods of introduction can also include rechargeable or biodegradable devices, particle acceleration devises (“gene guns”) and slow release polymeric devices. If desired, the compositions can be administered into a specific tissue, or into a blood vessel serving a specific tissue (e.g., the carotid artery to target brain). The pharmaceutical compositions can also be administered as part of a combinatorial therapy with other agents, either concurrently or in proximity (e.g., separated by hours, days, weeks, months). The activity of the compositions may be potentiated by other agents administered concurrently or in proximity.

The composition can be formulated in accordance with the routine procedures as a pharmaceutical composition adapted for administration to human beings or animals. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., which are, if desired, sterilized or mixed with auxiliary agents, e.g., preservatives, stabilizers, wetting agents, buffers or salts for influencing osmotic pressure, etc. The agent may be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

Agents described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

Representative methods incorporating delivery of an agent in a neoplasm-specific manner or in an angiogenesis-(neovascular)-specific manner are described below in relation to treatment, imaging, and diagnostics.

Therapy

In one embodiment of the invention, methods are available for treating neoplasias or other pathologies in an individual, by administering a therapeutic targeting agent. The term, “treatment” as used herein, can refer to ameliorating symptoms associated with the neoplasm or pathology; to reducing, preventing or delaying metastasis of the neoplasm; to reducing the number, volume, and/or size of one or more neoplasms; and/or to lessening the severity, duration or frequency of symptoms of the neoplasm or pathology. In these methods, a therapeutic targeting agent is used. A “therapeutic targeting agent,” as used herein, refers to an agent that targets neoplasm(s) or other pathologies for destruction (e.g., a chemotherapeutic agent), or otherwise treats the neoplasm, or reduces or eliminates the effects of neoplasm(s) or pathologies on the individual.

In another embodiment of the invention, methods are available for treating angiogenesis or the development of neovasculature, or other pathologies in an individual, by administering a therapeutic targeting agent. Representative additional conditions which can be treated using the methods described herein include atherosclerosis, diabetes and related sequelae, macular degeneration, heart disease (e.g., from ischemia), emphysema, chronic obstructive pulmonary disease, myocarditis, pulmonary and systemic hypertension and their sequelae, infection, and other conditions relating to expression of inflammatory-, angiogenesis- or neovasculature-related proteins, such as those described herein. Expression of angiogenesis-related proteins is a contributor to a variety of malignant, ischemic, inflammatory, infectious and immune disorders (see, e.g., Carmeleit, P., Nature Medicine 9(6):653-660 (2003); Carmeliet, P. and Jain, R., Nature 407:249-257 (2000)). Thus, the methods are similarly applicable to such conditions, which are collectively referred to herein as “pathology”.

The term, “treatment” as used herein, can refer to ameliorating symptoms associated with the angiogenesis, development of neovasculature, or other pathology; to reducing, preventing or delaying development of angiogenesis or of neovasculature; to reducing the number, volume, and/or size of one or more regions of angiogenesis or neovasculature; and/or to lessening the severity, duration or frequency of symptoms of the angiogenesis, neovasculature, or other pathology. Thus, a “therapeutic targeting agent,” as used herein, also refers to an agent that targets angiogenesis, development of neovasculature, or other pathologies for destruction (e.g., a chemotherapeutic agent), or otherwise treats angiogenesis, or reduces or eliminates negative effects of angiogenesis, neovasculature or other pathologies on the individual.

In a further embodiment of the invention, methods are available for enhancing or increasing angiogenesis or development of neovasculature in an individual, by administering an neovasculature targeting agent. A “neovasculature targeting agent,” as used herein, refers to an agent that enhances or increases angiogenesis or development of neovasculature, or which otherwise treats diseases or conditions which can be ameliorated by enhanced or increased angiogenesis or increased development of neovasculature.

In one embodiment, the therapeutic targeting agent or neovasculature targeting agent is or comprises an antibody that specifically binds a targeted protein, as described herein (e.g., VEGF receptor 1, VEGF receptor 2, Tie-2, aminopeptidase N, endoglin, C-CAM-1, neuropilin-1, AnnA1, AnnA8, EphA5, EphA7, myeloperoxidase, neucleolin, transferrin receptor, vitamin D binding receptor). An “antibody” is an immunoglobulin molecule obtained by in vitro or in vivo generation of the humoral response, and includes both polyclonal and monoclonal antibodies. The term also includes genetically engineered forms such as chimeric antibodies (e.g., humanized murine antibodies), heteroconjugate antibodies (e.g., bispecific antibodies), and recombinant single chain Fv fragments (scFv). The term “antibody” also includes multivalent antibodies as well as antigen binding fragments of antibodies, such as Fab′, F(ab′)₂, Fab, Fv, rIgG, and, inverted IgG, as well as the variable heavy and variable light chain domains. An antibody immunologically reactive with a targeted protein can be generated in vivo or by recombinant methods such as selection of libraries of recombinant antibodies in phage or similar vectors. See, e.g., Huse et al. (1989) Science 246:1275-1281; and Ward, et al. (1989) Nature 341:544-546; and Vaughan et al. (1996) Nature Biotechnology, 14:309-314. An “antigen binding fragment” includes any portion of an antibody that binds to the targeted protein. An antigen binding fragment may be, for example, a polypeptide including a CDR region, or other fragment of an immunoglobulin molecule which retains the affinity and specificity for the targeted protein.

In another embodiment, the therapeutic targeting agent is or comprises another agent that specifically binds to the targeted protein. Representative agents that specifically bind to a targeted protein include antibodies as described above, antibody-mimicking agents, and other specific binding partners as described above.

In yet another embodiment, the therapeutic targeting agent or neovasculature targeting agent comprises an active agent component and a targeting agent component. The targeting agent component is or comprises an agent that specifically binds to a targeted protein, as described above. In preferred embodiments of the invention, the targeting agent component specifically binds to a targeted protein that is associated with tumors, such as VEGF receptor 1, VEGF receptor 2, Tie-2, aminopeptidase N, endoglin, C-CAM-1, neuropilin-1, AnnA8, EphA5, EphA7, myeloperoxidase, nucleolin, transferrin receptor, AnnA1 or vitamin D binding protein, or a targeted protein that is associated with angiogenesis or with development of neovasculature. If desired, the targeting agent component can specifically bind to more than one targeted protein.

In one representative therapeutic targeting agent, a multivalent antibody is used. One moiety of the multivalent antibody can serve as the targeting agent component, and a second moiety of the multivalent antibody can serve as the active agent component.

In another embodiment, the targeting agent component is linked to the active agent component. For example, they can be covalently bonded directly to one another. Where the two are directly bonded to one another by a covalent bond, the bond may be formed by forming a suitable covalent linkage through an active group on each moiety. For instance, an acid group on one compound may be condensed with an amine, an acid or an alcohol on the other to form the corresponding amide, anhydride or ester, respectively. In addition to carboxylic acid groups, amine groups, and hydroxyl groups, other suitable active groups for forming linkages between a targeting agent component and an active agent component include sulfonyl groups, sulfhydryl groups, and the haloic acid and acid anhydride derivatives of carboxylic acids.

In other embodiments, the targeting agent component and an active agent component may be covalently linked to one another through an intermediate linker. The linker advantageously possesses two active groups, one of which is complementary to an active group on the targeting agent component, and the other of which is complementary to an active group on the active agent component. For example, where the both possess free hydroxyl groups, the linker may suitably be a diacid, which will react with both compounds to form a diether linkage between the two residues. In addition to carboxylic acid groups, amine groups, and hydroxyl groups, other suitable active groups for forming linkages between pharmaceutically active moieties include sulfonyl groups, sulfhydryl groups, and the haloic acid and acid anhydride derivatives of carboxylic acids.

Suitable linkers are set forth in the table below.

| FIRST | SECOND | |
|---|---|---|
| ACTIVE | ACTIVE | |
| GROUP | GROUP | SUITABLE LINKER |
| Amine | Amine | Diacid |
| Amine | Hydroxy | Diacid |
| Hydroxy | Amine | Diacid |
| Hydroxy | Hydroxy | Diacid |
| Acid | Acid | Diamine |
| Acid | Hydroxy | Amino acid, hydroxyalkyl |
| | | acid, sulfhydrylalkyl acid |
| Acid | Amine | Amino acid, hydroxyalkyl |
| | | acid, sulfhydrylalkyl acid |

Suitable diacid linkers include oxalic, malonic, succinic, glutaric, adipic, pimelic, suberic, azelaic, sebacic, maleic, fumaric, tartaric, phthalic, isophthalic, and terephthalic acids. While diacids are named, the skilled artisan will recognize that in certain circumstances the corresponding acid halides or acid anhydrides (either unilateral or bilateral) are preferred as linker reprodrugs. A preferred anhydride is succinic anhydride. Another preferred anhydride is maleic anhydride. Other anhydrides and/or acid halides may be employed by the skilled artisan to good effect.

Suitable amino acids include C-butyric acid, 2-aminoacetic acid, 3-aminopropanoic acid, 4-aminobutanoic acid, 5-aminopentanoic acid, 6-aminohexanoic acid, alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. Again, the acid group of the suitable amino acids may be converted to the anhydride or acid halide form prior to their use as linker groups.

Suitable diamines include 1,2-diaminoethane, 1,3-diaminopropane, 1,4-diaminobutane, 1,5-diaminopentane, 1,6-diaminohexane. Suitable aminoalcohols include 2-hydroxy-1-aminoethane, 3-hydroxy-1-aminoethane, 4-hydroxy-1-aminobutane, 5-hydroxy-1-aminopentane, 6-hydroxy-1-aminohexane. Suitable hydroxyalkyl acids include 2-hydroxyacetic acid, 3-hydroxypropanoic acid, 4-hydroxybutanoic acid, 5-hydroxypentanoic acid, 5-hydroxyhexanoic acid. The person having skill in the art will recognize that by selecting the components of the targeting agent component and active agent component having suitable active groups, and by matching them to suitable linkers, a broad palette of inventive compounds may be prepared within the scope of the present invention.

Moreover, the various linker groups can be designated either “weak” or “strong” based on the stability of the covalent bond which the linker functional group will form between the spacer and either the polar lipid carrier or the biologically active compound. The weak functionalities include, but are not limited to phosphoramide, phosphoester, carbonate, amide, carboxyl-phosphoryl anhydride, ester and thioester. The strong functionalities include, but are not limited to ether, thioether, amine, sterically hindered amides and esters. The use of a strong linker functional group between the spacer group and the biologically-active compound will tend to decrease the rate at which the compound will be released at the target site, whereas the use of a weak linker functional group between the spacer group and the compound may act to facilitate release of the compound at the target site.

Enzymatic release is also possible, but such enzyme-mediated modes of release will not necessarily be correlated with bond strength in such embodiments of the invention. Spacer moieties comprising enzyme active site recognition groups, such as spacer groups comprising peptides having proteolytic cleavage sites therein, are envisioned as being within the scope of the present invention. In certain embodiments, the linker moiety includes a spacer molecule which facilitated hydrolytic or enzymatic release of the active agent component from the targeting agent component. In particularly preferred embodiments, the spacer functional group is hydrolyzed by an enzymatic activity found in the target vascular tissue, preferably an esterase.

The active agent component, which is linked to the targeting agent component, can be or comprise any agent that achieves the desired therapeutic result, including agents such as the following, which can be used as an active agent component either for a therapeutic targeting agent or an neovasculature targeting agent, as appropriate: a radionuclide (e.g., I125, 123, 124, 131 or other radioactive agent); a chemotherapeutic agent (e.g., an antibiotic, antiviral or antifungal); an immune stimulatory agent (e.g., a cytokine); an anti-neoplastic agent: an anti-inflammatory agent; a pro-inflammatory agent; a pro-apoptotic agent (e.g., peptides or other agents to attract immune cells and/or stimulate the immune system); a pro-coagulant; a toxin (e.g., ricin, enterotoxin, LPS); an antibiotic; a hormone; a protein (e.g., a recombinant protein or a recombinant modified protein); a carrier protein (e.g., albumin, modified albumin); an enzyme; another protein (e.g., a surfactant protein, a clotting protein); a lytic agent; a small molecule (e.g., inorganic small molecules, organic small molecules, derivatives of small molecules, composite small molecules); aptamers; cells, including modified cells; vaccine-induced or other immune cells; nanoparticles (e.g, lipid or non-lipid based formulations, albumin-based formulations); transferring; immunoglobulins; multivalent antibodies; lipids; lipoproteins; lipopeptides; liposomes; lipid derivatives; an natural ligand; and altered protein (e.g., albumin or other blood carrier protein-based delivery system, modified to increase affinity for the targeted protein; orosomucoid); an agent that alters the extracellular matrix of the targeted cell; an agents that inhibits growth, migration or formation of vascular structures (for a therapeutic targeting agent); an agent that enhances or increases growth, migration or formation of vascular structures (for an neovasculature targeting agent); a gene or nucleic acid (e.g., an antisense oligonucleotide RNA; siRNA); viral or non-viral gene delivery vectors or systems; or a prodrug or promolecule.

For example, in one embodiment, a radionuclide or other radioactive agent can be used as the active agent component. The targeting agent component delivers the radioactive agent in a neoplasm-specific manner or a neovasculature-specific manner, allowing local radiation damage and resulting in radiation-induced apoptosis and necrosis throughout the neoplasm including in tumor cells, stromal calls, and endothelial cells of the neoplasm or throughout the area having unwanted angiogenesis or unwanted development of neovasculature. Alternatively, in another embodiment, an agent that stimulates angiogenesis or development of neovasculature can be used as the active agent component. The targeting agent component delivers the agent in a specific manner, resulting in increased angiogenesis or development of neovasculature at specific sites where the targeting agent binds.

In another particular embodiment, antisense oligonucleotides or other agents can be used as the active agent component, to alter, and particular to inhibit, production of a gene in a targeted tissue, such as a gene that is overexpressed in a tumor tissue (e.g., an oncogene or a gene associated with neoplasm, such as c-Jun, c-Fos, HER-2, E2F-1, RAS, FAS, NF, BRCA), or a gene that is overexpressed in angiogenesis. Alternatively, oligonucleotides or genes can be used to alter, and particularly to enhance, production of a protein in the targeted tissue, such as a gene that controls apoptosis or regulates cell growth; oligonucleotides or genes can also be used to produce a protein that is underexpressed or deleted in the targeted tissue, or to express a gene product that is directly or indirectly destructive to the neoplasm.

In a further particular embodiment, an anti-inflammatory agent can be used as the active agent. Representative agents include a non-steroidal anti-inflammatory agent; a steroidal or corticosteroidal anti-inflammatory agent; or other anti-inflammatory agent (e.g., histamine). In other embodiments, the active agent can be an agent to alter blood pressure (e.g., a diuretic, a vasopressin agonist or antagonist, angiotensin). Alternatively, pro-inflammatory agents can be used as active agents (e.g., to enhance angiogenesis or increase development of neovasculature, as described herein).

In another particular embodiment, chemotherapeutic agents for neoplastic diseases can be used as the active agent component. Representative agents include alkylating agents (nitrogen mustards, ethylenimines, alkyl sulfonates, nitrosoureas, and triazenes), antimetabolites (folic acid analogs such as methotrexate, pyrimidine analogs, and purine analogs), natural products and their derivatives (antibiotics, alkaloids, enzymes), hormones and antagonists (corticosteroids; adrenocorticosteroids, progestins, estrogens), and other similar agents. For example, in certain embodiments, the chemotherapeutic agent can be acytotoxic or cytostatic drugs. Chemotherapeutics may also include those which have other effects on cells such as reversal of the transformed state to a differentiated state or those which inhibit cell replication. Examples of known cytotoxic agents useful in the present invention are listed, for example, in Goodman et al., “The Pharmacological Basis of Therapeutics,” Sixth Edition, A. G. Gilman et a.l, eds./Macmillan Publishing Co. New York, 1980. These include taxol, nitrogen mustards, such as mechlorethamine, cyclophosphamide, melphalan, uracil mustard and chlorambucil; ethylenimine derivatives, such as thiotepa; alkyl sulfonates, such as busulfan; nitrosoureas, such as carmustine, lomustine, semustine and streptozocin; triazenes, such as dacarbazine; folic acid analogs, such as methotrexate; pyrimidine analogs, such as fluorouracil, cytarabine and azaribine; purine analogs, such as mercaptopurine and thioguanine; vinca alkaloids, such as vinblastine and vincristine; antibiotics, such as dactinomycin, daunorubicin, doxorubicin, bleomycin, mithramycin and mitomycin; enzymes, such as L-asparaginase; platinum coordination complexes, such as cisplatin; substituted urea, such as hydroxyurea; methyl hydrazine derivatives, such as procarbazine; adrenocortical suppressants, such as mitotane; hormones and antagonists, such as adrenocortisteroids (prednisone), progestins (hydroxyprogesterone caproate, medroprogesterone acetate and megestrol acetate), estrogens (diethylstilbestrol and ethinyl estradiol), antiestrogens (tamoxifen), and androgens (testosterone propionate and fluoxymesterone).

Drugs that interfere with intracellular protein synthesis can also be used; such drugs are known to these skilled in the art and include puromycin, cycloheximide, and ribonuclease.

Most of the chemotherapeutic agents currently in use in treating cancer possess functional groups that are amenable to chemical crosslinking directly with an amine or carboxyl group of a targeting agent component. For example, free amino groups are available on methotrexate, doxorubicin, daunorubicin, cytosinarabinoside, cis-platin, vindesine, mitomycin and bleomycin while free carboxylic acid groups are available on methotrexate, melphalan, and chlorambucil. These functional groups, that is free amino and carboxylic acids, are targets for a variety of homobifunctional and heterobifunctional chemical crosslinking agents which can crosslink these drugs directly to a free amino group.

Peptide and polypeptide toxins are also useful as active agent components, and the present invention specifically contemplates embodiments wherein the active agent component is a toxin. Toxins are generally complex toxic products of various organisms including bacteria, plants, etc. Examples of toxins include but are not limited to: ricin, ricin A chain (ricin toxin), Pseudomonas exotoxin (PE), diphtheria toxin (DT), Clostridium perfringens phospholipase C (PLC), bovine pancreatic ribonuclease (BPR), pokeweed antiviral protein (PAP), abrin, abrin A chain (abrin toxin), cobra venom factor (CVF), gelonin (GEL), saporin (SAP), modeccin, viscumin and volkensin.

The present invention also contemplates dyes used, for example, in photodynamic therapy, and used in conjunction with appropriate non-ionizing radiation. The use of light and porphyrins in methods of the present invention is also contemplated and their use in cancer therapy has been reviewed. van den Bergh, Chemistry in Britain, 22: 430-437 (1986), which is incorporated herein in its entirety by reference.

In a further particular embodiment, an anti-inflammatory agent can be used as the active agent. Representative agents include a non-steroidal anti-inflammatory agent; a steroidal or corticosteroidal anti-inflammatory agent; or other anti-inflammatory agent (e.g., histamine). Alternatively, pro-inflammatory agents can be used as active agents (e.g., to enhance angiogenesis or increase development of neovasculature, as described herein).

Prodrugs or promolecules can also be used as the active agent. For example, a prodrug that is used as an active agent can subsequently be activated (converted) by administration of an appropriate enzyme, or by endogenous enzyme in the targeted tissue. Alternatively, the activating enzyme can be co-administered or subsequently administered as another active agent as part of a therapeutic agent as described herein; or the prodrug or promolecule can be activated by a change in pH to a physiological pH upon administration. Representative prodrugs include Herpes simplex virus thymidine kinase (HSV TK) with the nucleotide analog GCV; cytosine deaminase ans t-fluorocytosine; alkaline phosphatase/etoposidephosphate; and other prodrugs (e.g., those described in Greco et al., J. Cell. Phys. 187:22-36, 2001; and Konstantinos et al., Anticancer Research 19:605-614, 1999; see also Connors, T. A., Stem Cells 13(5): 501-511, 1995; Knox, R. J., Baldwin, A. et al., Arch. Biochem. Biophys. 409(1):197-206, 2003; Syrigos, K. N. and Epenetos, A. A., Anticancer Res. 19(1A): 605-613, 1999; Denny, W. A., JBB 1:48-70, 2003).

In another embodiment of the invention, the targeting agent component and/or the active agent component comprises a chelate moiety for chelating a metal, e.g., a chelator for a radiometal or paramagnetic ion. In preferred embodiments, the a chelator is a chelator for a radionuclide. Radionuclides useful within the present invention include gamma-emitters, positron-emitters, Auger electron-emitters, X-ray emitters and fluorescence-emitters, with beta- or alpha-emitters preferred for therapeutic use. Examples of radionuclides useful as toxins in radiation therapy include: ³²P, ³³P, ⁴³K, ⁴⁷Sc, ⁵²Fe, ⁵⁷Co, ⁶⁴Cu, ⁶⁷Ga, ⁶⁷Cu, ⁶⁸Ga, ⁷¹Ge, ⁷⁵Br, ⁷⁶Br, ⁷⁷Br, ⁷⁷As, ⁷⁷Br, ⁸¹Rb/^{81M}Kr, ^{87M}Sr, ⁹⁰Y, ⁹⁷Ru, ⁹⁹Tc, ¹⁰⁰Pd, ¹⁰¹Rh, ¹⁰³Pb, ¹⁰⁵Rh, ¹⁰⁹Pd, ¹¹¹Ag, ¹¹¹In, ¹¹³In, ¹¹⁹Sb ¹²¹Sn, ¹²³I, ¹²⁵I, ¹²⁷Cs, ¹²⁸Ba, ¹²⁹Cs, ¹³¹I, ¹³¹Cs, ¹⁴³Pr, ¹⁵³Sm, ¹⁶¹Tb, ¹⁶⁶Ho, ¹⁶⁹Eu, ¹⁷⁷Lu, ¹⁸⁶Re, ¹⁸⁸Re, ¹⁸⁹Re, ¹⁹¹Os, ¹⁹³Pt, ¹⁹⁴Ir, ¹⁹⁷Hg, ¹⁹⁹Au, ²⁰³Pb, ²¹¹At, ²¹²Pb, ²¹²Bi and ²¹³Bi. Preferred therapeutic radionuclides include ¹⁸⁸Re, ¹⁸⁶Re, ²⁰³Pb, ²¹²Pb, ²¹²Bi, ¹⁰⁹Pd, ⁶⁴Cu, ⁶⁷Cu, ⁹⁰Y, ¹²⁵I, ¹³¹I, ⁷⁷Br, ²¹¹At, ⁹⁷Ru, ¹⁰⁵Rh, ¹⁹⁸ Au and ¹⁹⁹Ag, ¹⁶⁶Ho or ¹⁷⁷Lu. Conditions under which a chelator will coordinate a metal are described, for example, by Gansow et al., U.S. Pat. Nos. 4,831,175, 4,454,106 and 4,472,509.

In one embodiment, for example, ^{99m}Tc can be used as a radioisotope for therapeutic and diagnostic applications (as described below), as it is readily available to all nuclear medicine departments, is inexpensive, gives minimal patient radiation doses, and has ideal nuclear imaging properties. It has a half-life of six hours which means that rapid targeting of a technetium-labeled antibody is desirable. Accordingly, in certain preferred embodiments, the therapeutic targeting agent includes a chelating agents for technium.

The therapeutic targeting agent can also comprise radiosensitizing agents, e.g., a moiety that increase the sensitivity of cells to radiation. Examples of radiosensitizing agents include nitroimidazoles, metronidazole and misonidazole (see: DeVita, V. T. Jr. in Harrison's Principles of Internal Medicine, p. 68, McGraw-Hill Book Co., N.Y. 1983, which is incorporated herein by reference). The therapeutic targeting agent that comprises a radiosensitizing agent as the active moiety is administered and localizes in the endothelial call and/or in any other cells of the neoplasm. Upon exposure of the individual to radiation, the radiosensitizing agent is “excited” and causes the death of the cell.

There are a wide range of moieties which can serve as chelating ligands and which can be derivatized as part of the therapeutic targeting agent. For instance, the chelating ligand can be a derivative of 1,4,7,10-tetraazacyclododecanetetraacetic acid (DOTA), ethylenediaminetetraacetic acid (EDTA), diethylenetriaminepentaacetic acid (DTPA) and 1-p-Isothiocyanato-benzyl-methyl-diethylenetriaminepentaacetic acid (ITC-MX). These chelators typically have groups on the side chain by which the chelator can be used for attachment to a targeting agent component. Such groups include, e.g., benzylisothiocyanate, by which the DOTA, DTPA or EDTA can be coupled to, e.g., an amine group of the inhibitor.

In one embodiment, the agent is an “NₓS_{y}” chelate moiety. As defined herein, the term “NₓS_{y} chelates” includes bifunctional chelators that are capable of coordinately binding a metal or radiometal and, preferably, have N₂S₂ or N₃S cores. Exemplary NₓS_{y} chelates are described, e.g., in Fritzberg et al. (1988) PNAS 85:4024-29; and Weber et al. (1990) Bioconjugate Chem. 1:431-37; and in the references cited therein. The Jacobsen et al. PCT application WO 98/12156 provides methods and compositions, i.e. synthetic libraries of binding moieties, for identifying compounds which bind to a metal atom. The approach described in that publication can be used to identify binding moieties which can subsequently be incorporated into therapeutic targeting agents.

A problem frequently encountered with the use of conjugated proteins in radiotherapeutic and radiodiagnostic applications is a potentially dangerous accumulation of the radiolabeled moiety fragments in the kidney. When the conjugate is formed using a acid- or base-labile linker, cleavage of the radioactive chelate from the protein can advantageously occur. If the chelate is of relatively low molecular weight, it is not retained in the kidney and is excreted in the urine, thereby reducing the exposure of the kidney to radioactivity. However, in certain instances, it may be advantageous to utilize acid- or base-labile linkers in the subject ligands for the same reasons they have been used in labeled proteins.

Other appropriate active agents include agents that induce intravascular coagulation, or which damage the endothelium, thereby causing coagulation and effectively infracting a neoplasm or other targeted pathology. In addition, if desired, enzymes activated by other agents (e.g., biotin, activated by avidin) can be used as active agents or as part of the therapeutic targeting agent.

The therapeutic targeting agents can be synthesized, by standard methods known in the art (e.g., by recombinant DNA technology or other means), to provide reactive functional groups which can form acid-labile linkages with, e.g., a carbonyl group of the ligand. Examples of suitable acid-labile linkages include hydrazone and thiosemicarbazone functions. These are formed by reacting the oxidized carbohydrate with chelates bearing hydrazide, thiosemicarbazide, and thiocarbazide functions, respectively. Alternatively, base-cleavable linkers, which have been used for the enhanced clearance of the radiolabel from the kidneys, can be used. See, for example, Weber et al. 1990 Bioconjug. Chem. 1:431. The coupling of a bifunctional chelate via a hydrazide linkage can incorporate base-sensitive ester moieties in a linker spacer arm. Such an ester-containing linker unit is exemplified by ethylene glycolbis(succinimidyl succinate), (EGS, available from Pierce Chemical Co., Rockford, Ill.), which has two terminal N-hydroxysuccinimide (NHS) ester derivatives of two 1,4-dibutyric acid units, each of which are linked to a single ethylene glycol moiety by two alkyl esters. One NHS ester may be replaced with a suitable amine-containing BFC (for example 2-aminobenzyl DTPA), while the other NHS ester is reacted with a limiting amount of hydrazine. The resulting hyrazide is used for coupling to the targeting agent component, forming an ligand-BFC linkage containing two alkyl ester functions. Such a conjugate is stable at physiological pH, but readily cleaved at basic pH.

Therapeutic targeting agents labeled by chelation are subject to radiation-induced scission of the chelator and to loss of radioisotope by dissociation of the coordination complex. In some instances, metal dissociated from the complex can be re-complexed, providing more rapid clearance of non-specifically localized isotope and therefore less toxicity to non-target tissues. For example, chelator compounds such as EDTA or DTPA can be infused into patients to provide a pool of chelator to bind released radiometal and facilitate excretion of free radioisotope in the urine.

In still other embodiments, a Boron addend, such as a carborane, can be used. For example, carboranes can be prepared with carboxyl functions on pendant side chains, as is well known in the art. Attachment of such carboranes to an amine functionality, e.g., as may be provided on the targeting agent component can be achieved by activation of the carboxyl groups of the carboranes and condensation with the amine group to produce the conjugate. Such therapeutic agents can be used for neutron capture therapy.

In a further embodiment, RNAi is used. “RNAi construct” is a generic term used throughout the specification to include small interfering RNAs (siRNAs), hairpin RNAs, and other RNA species which can be delivered ectopically to a cell, cleaved by the enzyme dicer and cause gene silencing in the cell. The term “small interfering RNAs” or “siRNAs” refers to nucleic acids around 19-30 nucleotides in length, and more preferably 21-23 nucleotides in length. The siRNAs are double-stranded, and may include short overhangs at each end. Preferably, the overhangs are 1-6 nucleotides in length at the 3′ end. It is known in the art that the siRNAs can be chemically synthesized, or derive by enzymatic digestion from a longer double-stranded RNA or hairpin RNA molecule. For efficiency, an siRNA will generally have significant sequence similarity to a target gene sequence. Optionally, the siRNA molecules includes a 3′ hydroxyl group, though that group may be modified with a fatty acid moiety as described herein. The phrase “mediates RNAi” refers to (indicates) the ability of an RNA molecule capable of directing sequence-specific gene silencing, e.g., rather than a consequence of induction of a sequence-independent double stranded RNA response, e.g., a PKR response.

In certain embodiments, the RNAi construct used for the active agent component is a small-interfering RNA (siRNA), preferably being 19-30 base pairs in length. Alternatively, the RNAi construct is a hairpin RNA which can be processed by cells (e.g., is a dicer substrate) to produce metabolic products in vivo in common with siRNA treated cells, e.g., a processed to short (19-22 mer) guide sequences that induce sequence specific gene silencing. In a preferred embodiment, the treated animal is a human.

The RNAi constructs contain a nucleotide sequence that hybridizes under physiologic conditions of the cell to the nucleotide sequence of at least a portion of the mRNA transcript for the gene to be inhibited (i.e., the “target” gene). The double-stranded RNA need only be sufficiently similar to natural RNA that it has the ability to mediate RNAi. Thus, the invention has the advantage of being able to tolerate sequence variations that might be expected due to genetic mutation, strain polymorphism or evolutionary divergence. The number of tolerated nucleotide mismatches between the target sequence and the RNAi construct sequence is no more than 1 in 5 basepairs, or 1 in 10 basepairs, or 1 in 20 basepairs, or 1 in 50 basepairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition.

Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 50°C. or 70° C. hybridization for 12-16 hours; followed by washing).

Production of RNAi constructs can be carried out by chemical synthetic methods or by recombinant nucleic acid techniques. Endogenous RNA polymerase of the treated cell may mediate transcription in vivo, or cloned RNA polymerase can be used for transcription in vitro.

The RNAi constructs may include other modifications, such as to the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of a nitrogen or sulfur heteroatom. Modifications in RNA structure may be tailored to allow specific genetic inhibition while avoiding a general cellular response to dsRNA (a “PKR-mediated response”). Likewise, bases may be modified to block the activity of adenosine deaminase. The RNAi construct may be produced enzymatically or by partial/total organic synthesis, any modified ribonucleotide can be introduced by in vitro enzymatic or organic synthesis.

Methods of chemically modifying other RNA molecules can be adapted for modifying RNAi constructs (see, for example, Heidenreich et al. (1997) Nucleic Acids Res, 25:776-780; Wilson et al. (1994) J Mol Recog 7:89-98; Chen et al. (1995) Nucleic Acids Res 23:2661-2668; Hirschbein et al. (1997) Antisense Nucleic Acid Drug Dev 7:55-61). Merely to illustrate, the backbone of an RNAi construct can be modified with phosphorothioate, phosphorodithioate, methylphosphonate, chimeric methylphosphonate-phosphodiesters, phosphoramidate, boranophosphate, phosphotriester, formacetal, 3′-thioformacetal, 5′-thioformacetal, 5′-thioether, carbonate, 5′-N-carbamate, sulfate, sulfonate, sulfamate, sulfonamide, sulfone, sulfite, sulfoxide, sulfide, hydroxylamine, methylene(methylimino) (MMI), methyleneoxy(methylimino) (MOMI) linkages, peptide nucleic acids, 5-propynyl-pyrimidine containing oligomers or sugar modifications (e.g., 2′-substituted ribonucleosides, a-configuration).

The double-stranded structure may be formed by a single self-complementary RNA strand or two complementary RNA strands. RNA duplex formation may be initiated either inside or outside the cell.

In certain embodiments, to reduce unwanted immune stimulation, the RNAi construct is designed so as not to include unmodified cytosines occurring 5′ to guanines, e.g., to avoid stimulation of B cell mediated immunosurveillance.

In certain embodiments in which the RNAi is to be delivered for local therapeutic effect, the backbone linkages can be chosen so as titrate the nuclease sensitivity to make the RNAi sufficiently nuclease resistant to be effective in the tissue of interest (e.g., the neoplasm), but not so nuclease resistant that significant amounts of the construct could escape the tissue undegraded. With the use of this strategy, RNAi constructs are available for gene silencing in the tissue of interest, but are degraded before they can enter the wider circulation.

The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition.

In certain embodiments, the subject RNAi constructs are siRNAs. These nucleic acids are around 19-30 nucleotides in length, and even more preferably 21-23 nucleotides in length, e.g., corresponding in length to the fragments generated by nuclease “dicing” of longer double-stranded RNAs. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA molecules comprise a 3′ hydroxyl group.

The siRNA molecules of the present invention can be obtained using a number of techniques known to those of skill in the art. For example, the siRNA can be chemically synthesized or recombinantly produced using methods known in the art. For example, short sense and antisense RNA oligomers can be synthesized and annealed to form double-stranded RNA structures with 2-nucleotide overhangs at each end (Caplen, et al. (2001) Proc Natl Acad Sci USA, 98:9742-9747; Elbashir, et al. (2001) EMBO J, 20:6877-88). These double-stranded siRNA structures can then be directly introduced to cells, either by passive uptake or a delivery system of choice, such as described below.

In certain embodiments, the siRNA constructs can be generated by processing of longer double-stranded RNAs, for example, in the presence of the enzyme dicer. In one embodiment, the Drosophila in vitro system is used. In this embodiment, dsRNA is combined with a soluble extract derived from Drosophila embryo, thereby producing a combination. The combination is maintained under conditions in which the dsRNA is processed to RNA molecules of about 21 to about 23 nucleotides.

The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

Modification of siRNA molecules with fatty acids can be carried out at the level of the precursors, or, perhaps more practically, after the RNA has been synthesized. The latter may be accomplished in certain instances using nucleoside precursors in the synthesis of the polymer that include functional groups for formation of the linker-fatty acid moiety.

In certain preferred embodiments, at least one strand of the siRNA molecules has a 3′ overhang from about 1 to about 6 nucleotides in length, though may be from 2 to 4 nucleotides in length. More preferably, the 3′ overhangs are 1-3 nucleotides in length. In certain embodiments, one strand having a 3′ overhang and the other strand being blunt-ended or also having an overhang. The length of the overhangs may be the same or different for each strand. In order to further enhance the stability of the siRNA, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythyinidine is tolerated and does not affect the efficiency of RNAi. The absence of a 2′ hydroxyl significantly enhances the nuclease resistance of the overhang in tissue culture medium and may be beneficial in vivo.

In other embodiments, the RNAi construct is in the form of a long double-stranded RNA. In certain embodiments, the RNAi construct is at least 25, 50, 100, 200, 300 or 400 bases. In certain embodiments, the RNAi construct is 400-800 bases in length. The double-stranded RNAs are digested intracellularly, e.g., to produce siRNA sequences in the cell. However, use of long double-stranded RNAs in vivo is not always practical, presumably because of deleterious effects which may be caused by the sequence-independent dsRNA response. In such embodiments, the use of local delivery systems and/or agents which reduce the effects of interferon or PKR are preferred.

In certain embodiments, the RNAi construct is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci USA, 2002, 99:6047-52). Preferably, such hairpin RNAs are engineered in cells or in an animal to ensure continuous and stable suppression of a desired gene. It is known in the art that siRNAs can be produced by processing a hairpin RNA in the cell.

The therapeutic targeting agent, alone or in a composition, is administered in a therapeutically effective amount, which is the amount used to treat the neoplasm or to treat angiogenesis or unwanted development of neovasculature. The amount which will be therapeutically effective will depend on the nature of the neoplasm, neovasculature or angiogenesis, the extent of disease and/or metastasis, and other factors, and can be determined by standard clinical techniques. In addition, in vitro or in vivo assays may optionally be employed to help identify optimal dosage ranges. The precise dose to be employed in the formulation will also depend on the route of administration, and the seriousness of the symptoms, and should be decided according to the judgment of a practitioner and each patient's circumstances. Effective doses may be extrapolated from dose-response curves derived from in vitro or animal model test systems.

Although the embodiments above describe treatment of undesirable neovasculature or angiogenesis or other pathologies, the methods are also applicable to situations in which angiogenesis or neovasculature is desirable (e.g., regrowth of blood vessels after reattachment of a previously severed body part; development of blood vessels to compensate for damaged blood vessels after myocardial infarction; or for other injury or disease which is treated by improving blood flow, tissue repair, neovasculature development and/or angiogenesis). In this embodiment, the neovasculature targeting agent comprises a compound (e.g., as the active agent component) that enhances angiogenesis or development of neovasculature. The term, “treatment” as used in this specific embodiment, refers to enhancing or increasing angiogenesis or to increasing development of neovasculature.

In addition, in a further embodiment of the invention, the targeted proteins described herein can be used as focal point for immune stimulation, in order to effect immune attack by a patient's own immune system against the targeted agent. In one embodiment, cells can be modified to produce a targeted protein: for example, dendritic cells from an individual can be isolated, and then inoculated with a targeted protein or an antigenic fragment of the targeted protein, and then the dendritic cells can be readministered to the individual to initiate an immune attack against the targeted protein. In addition, T cells specific for target protein or fragments thereof, including cells induced by vaccination, can be isolated and used directly to attack the neoplasm immunologically. Alternatively, a therapeutic targeting agent comprising a targeted protein expressed on endothelial cell surface can be administered to generate immune response. Other standard techniques for stimulating immune system attack can be used as well. In this manner, ‘personalized medicine’ for each patient can be designed, to target the particular individual's neoplasm or other pathology. Thus, a method of treating neoplasia in an individual by administering to the individual a therapeutic targeting agent that comprises a targeted protein expressed on endothelial cell surface, and generating an immune response against the targeted protein, is now available.

Imaging In Vivo and Diagnostics

The present invention also relates to methods of delivering imaging agents in a neoplasm-specific manner, for physical imaging, e.g., for use in assessing an individual for the presence of neoplasia, including primary and/or secondary (metastatic) neoplasms, as well as to the use of the described agents for manufacture of medicaments for use in physical imaging both in vivo and in vitro. In the methods of the invention, the imaging agent is delivered to, into and/or across vascular endothelium in a neoplasm-specific manner through an agent of interest. “Neoplasm-specific” indicates that the agent preferentially or selectively binds to a neoplasm. The present invention also relates to methods of delivering imaging agents in a neovasculature-specific manner, for physical imaging, e.g., for use in assessing an individual for the presence of angiogenesis or of neovasculature. In the methods of the invention, the imaging agent is delivered to, into and/or across vascular endothelium in a neovasculature-specific manner through an agent of interest. “Neovasculature-specific” indicates that the agent preferentially or selectively binds to new blood vessel growth. It is noted that new blood vessels, “neovasculature,” may be in varying stages of development and at different stages of maturity; for the purposes of this application, “neovasculature” refers to new blood vessel growth that differs from normal vasculature, either in stage, maturity, or other relevant characteristic.

In the methods of the invention, an “imaging agent” is used. The imaging agent comprises a targeting agent component and an imaging agent component. The targeting agent component can be neoplasm-specific, and specifically binds to a targeted protein expressed on neoplasm endothelial cell surface (e.g., to a targeted protein as described above). The imaging agent component (comprising the imaging agent, and, if necessary, other components such as a means to couple the imaging agent component to the targeting agent component) can be, for example, a radioactive agent (e.g., radioiodine (125I, 131I); technetium; yttrium; 35S or 3H) or other radioisotope or radiopharmaceutical; a contrast agent (e.g., gadolinium; manganese; barium sulfate; an iodinated or noniodinated agent; an ionic agent or nonionic agent); a magnetic agent or a paramagnetic agent (e.g., gadolinium, iron-oxide chelate); liposomes (e.g., carrying radioactive agents, contrast agents, or other imaging agents); nanoparticles; ultrasound agents (e.g., microbubble-releasing agents); a gene vector or virus inducing a detecting agent (e.g., including luciferase or other fluorescent polypeptide); an enzyme (horseradish peroxidase, alkaline phosphatase, β-galactosidase, or acetylcholinesterase); a prosthetic group (e.g., streptavidin/biotin and avidin/biotin); a fluorescent material (e.g., umbelliferone, fluorescein, fluorescein isothiocyanate, rhodamine, dichlorotriazinylamine fluorescein, dansyl chloride or phycoerythrin); a luminescent material (e.g., luminol); a bioluminescent material (e.g., luciferase, luciferin, aequorin); or any other imaging agent that can be employed for imaging studies (e.g., for CT, fluoroscopy, SPECT imaging, optical imaging, PET, MRI, gamma imaging).

The imaging agent can be used in methods of performing physical imaging of an individual. “Physical imaging,” as used herein, refers to imaging of all or a part of an individual's body (e.g., by the imaging studies methods set forth above). Physical imaging can be “positive,” that is, can be used to detect the presence of a specific type of tissue or pathology (e.g., angiogenesis, neovasculature). For example, in one embodiment, positive physical imaging can be used to detect the presence or absence of a neoplasm, including the presence or absence of metastases, or to assess an individual for the presence or absence, or extent, or angiogenesis or of neovasculature. Alternatively, in another embodiment, positive physical imaging can be used to detect the presence or absence of a normal (non-disease) tissue, such as the presence of or absence of an organ. Alternatively, the physical imaging can be “negative,” that is, can be used to detect the absence of a specific type of tissue. For example, in one embodiment, negative physical imaging can be used to detect the absence or presence of a normal tissue, where the absence is indicative of a loss of function consistent with a pathology. Both positive and negative physical imaging permit visualization and/or detection of both normal and of abnormal pathology, and can be used to quantify or determine the extent, size, and/or number of an organ or of a type of neoplasm, as well as to quantify or determine the extent of angiogenesis or of neovasculature. Thus, an estimate can be made of the extent of disease or of angiogenesis or neovasculature, facilitating, for example, clinical diagnosis and/or prognosis.

For physical imaging, an imaging agent is administered to the individual. These methods of physical imaging can be used, for example, to assess an individual for the presence or absence, or extent, of neoplasia (e.g., by “positive” imaging as described above). In these embodiments, the targeting agent component binds to or localizes to a targeted protein that is associated with a neoplasm (e.g., a targeted protein that is present on the vascular endothelium of neoplasm; or a targeted protein that is expressed to a greater degree in a neoplastic tissue than in a comparable normal tissue). The agent of interest is administered to the individual (e.g., intravenously); upon administration, the targeted imaging agents can be visualized noninvasively by conventional external detection means (designed for the imaging agent), to detect the preferential or specific accumulation of a concentration of the agent of interest in the neoplasm. A “concentration,” as used herein, is an amount of the agent of interest at a particular location in the individual's body that is greater than would be expected from mere circulation or diffusion of the agent of interest in the individual, or that is greater than would be expected in a comparable normal tissue in the individual. A concentration is indicative of binding of the agent of interest to the neoplasm or to new blood vessels, and thus is indicative of the presence of the neoplasm or of angiogenesis or neovasculature. These methods can be used to assess an individual for the presence or absence not only of primary neoplasms, but also of metastases, as well as for angiogenesis or neovasculature. Representative new blood vessel growth includes, for example, growth related to a variety of diseases, including, for example, atherosclerosis, macular degeneration or diabetic retinopathy, or acute or chronic inflammation. In another embodiment of imaging in vivo, an imaging agent as described herein can be used to facilitate imaging-assisted therapy, such as surgical removal of a neoplasm or surgical removal of undesirable new blood vessel growth.

In other embodiments, the methods can be used to assess an individual for the presence or absence of normal (non-disease) function of an organ or bodily system (e.g., by “negative” imaging as described above). In these embodiments, the targeting agent component binds to and localizes to a targeted protein present in the normal tissue but not in the pathologic (abnormal) tissue. The agent is administered to the to the individual, and then the individual is assessed for the absence (or presence) of the agent of interest. An absence of the imaging agent where it is expected in the structures targeted by the targeting agent component, in combination with the presence of the agent of interest in other parts of the structures targeted by the targeting agent component, is indicative of a loss of function that is consistent with the presence of pathology.

In another embodiment of imaging in vivo, an imaging agent as described herein can be used to facilitate surgical removal of a neoplasm or to facilitate surgical removal of undesirable new blood vessel growth. For example, an imaging agent, such as an imaging agent that comprises a luminescent component, is administered to an individual in a manner such that the imaging agent targets neoplasm(s) or new blood vessel growth in the individual. A surgeon can then identify the presence of the imaging agent (through luminescence, for example), and is more easily able to remove neoplasm tissue or blood vessel growth (angiogenic tissue) that has thus been tagged with the imaging age

Furthermore, the growth, regression, or metastasis of a neoplasm, as well as the growth or regression of new blood vessels, can be assessed by serial imaging of an individual in this manner; each imaging session provides a view of the extent, size, location and/or number of neoplasm(s) or of new blood vessels.

If desired, the imaging agent can further comprise a therapeutic agent. A “therapeutic agent,” as used herein, refers to an agent that targets neoplasm(s), new blood vessels (angiogenic tissue), or other pathologies for destruction (e.g., a chemotherapeutic agent) or otherwise reduces or eliminates the effects of neoplasm(s) or angiogenic tissue, or pathologies on the individual. Additional uses of therapeutic agents are discussed above in relation to therapy.

In preferred embodiments of the invention, the targeting agent component specifically binds to a targeted protein that is associated with neoplasms, such as VEGF receptor 1, VEGF receptor 2, Tie-2, aminopeptidase N, endoglin, C-CAM-1, neuropilin-1, AnnA8, EphA5, EphA7, myeloperoxidase, nucleolin, transferrin receptor, AnnA1 and vitamin D binding protein; and the imaging agent is used for imaging of neoplasias or new blood vessels.

Although the embodiments above describe imaging of undesirable angiogenesis or neovasculature, the methods are equally applicable to situations in which angiogenesis or development of neovasculature is desirable (e.g., as described above in relation to treatment). In these methods, angiogenesis or development of neovasculature is similarly assessed by administration of an imaging agent as described above. If desired, the imaging agent can further comprise a therapeutic agent such as an neovasculature targeting agent, which enhances/increases angiogenesis or neovasculature, as discussed above in relation to therapy.

Imaging Ex Vivo and Diagnostics

In another embodiment, the present invention relates to methods of delivering imaging agents in a neoplasm-specific manner or a neovasculature-specific manner, for use ex vivo, e.g., for analysis of a tissue sample or cell sample. The term, “tissue sample,” as used herein, refers not only to a sample from tissue (e.g., skin, brain, breast, lung, kidney, prostate, ovarian, head and neck, liver, or other organ), but also to a blood sample. The tissue can be normal tissue, benign or malignant, or a combination thereof (e.g., a biopsy sample), and comprise a tissue for which the status (normal, benign or malignant) is unknown.

In one embodiment of the invention, an imaging agent, as described above, is used to perform ex vivo imaging. “Ex vivo imaging,” as used herein, refers to imaging of a tissue sample or cell sample that has been removed from an individual's body (e.g., by surgical removal of a tissue sample such as a neoplasm sample, or a cell sample; by venipuncture; or other means). The imaging permits visualization and/or detection of abnormal pathology (e.g., neoplasm or angiogenic tissue), and can be used to quantify or determine the extent, size, location and/or number of a type of neoplasm(s) or new blood vessel growth in a sample. Thus, an estimate can be made of the extent of disease, facilitating, for example, clinical diagnosis and/or prognosis.

In one embodiment, for ex vivo imaging, the imaging agent is administered to an individual as described above. A biopsy sample can then be taken from the individual, and the biopsy sample can then be assessed for the presence or absence of a concentration of the agent of interest. Alternatively, in another embodiment of ex vivo imaging, the imaging agent as described above is applied to the tissue sample. The tissue sample can then be assessed for the presence or absence of a concentration of the agent of interest. A “concentration,” as used herein, is an amount of the agent of interest that is greater than would be expected from mere diffusion of the agent of interest in the tissue sample. A concentration is indicative of binding of the agent of interest, and thus is indicative of the presence of neoplasm or neoplasm or new blood vessel growth (angiogenesis or development of neovasculature). These methods can be used to assess a tissue sample to determine whether a neoplasm is malignant (i.e., demonstrates a concentration of the imaging agent, corresponding to a concentration of a neoplasm-specific protein) or benign, or whether there is a presence of new blood vessel growth. In a preferred embodiment, the tissue sample used for ex vivo imaging is a biopsy sample.

Although the embodiments above describe imaging of undesirable angiogenesis or neovasculature ex vivo, the methods are equally applicable to situations in which angiogenesis or development of neovasculature is desirable, as described above in relation to treatment and in vivo imaging.

Molecular Signature and Diagnostics

In view of the identification of a set of neoplasm-specific target proteins, methods are also now available to assess a tissue sample for a molecular signature of a neoplasm. The molecular signature comprises the expression of more than one of the targeted proteins described herein (e.g., AnnA1, AnnA8, EphA5, EphA7, myeloperoxidase, nucleolin, transferrin receptor, vitamin D binding receptor, VEGF receptor 1, VEGF receptor 2, Tie-2, aminopeptidase N, endoglin, C-CAM-1, and neuropilin). A tissue sample can be assessed for the presence of some or all of these proteins; the presence of the proteins is indicative of neoplasm endothelium. An assessment can also be made of the aggressiveness of a neoplasm; an increased number of targeted proteins in the molecular signature is indicative of aggressive disease and also is indicative of poorer prognosis. The invention also comprises kits for use in assessing a sample for a neoplasm molecular signature, comprising, for example, agents (e.g., antibodies, labeled antibodies) to facilitate identification of the presence of one or more targeted proteins.

Assessment of Treatment Efficacy and Prognosis

The in vitro and/or ex vivo diagnosis methods described above can be used in methods for assessment of treatment efficacy in a patient. Thus, the current invention also pertains to methods of monitoring the response of an individual to treatment with a therapeutic agent, such as a therapeutic targeting agent, as described above, or other therapeutic agent, as well as to determine the efficacy of treatment, by comparing the quantity, extent, size, location and/or number of neoplasms, or the quantity or extent of angiogenesis or of neovasculature, both before and during or after treatment.

In one embodiment, ex vivo analysis can be performed to assess treatment efficacy in a patient. Thus, the current invention also pertains to methods of monitoring the response of an individual to treatment with a therapeutic targeting agent, as described above, or other therapeutic agent.

For example, in one aspect of the invention, an individual can be assessed for response to treatment with an therapeutic targeting agent or other therapeutic agent, by examining the level of the targeted protein in different tissues, cells and/or body fluids of the individual. Blood, serum, plasma or urinary levels of the targeted protein, or ex vivo production of the targeted protein, can be measured before, and during or after treatment with the therapeutic targeting agent or other therapeutic agent, as can levels of the targeted protein in tissues. The level before treatment is compared with the level during or after treatment. The efficacy of treatment is indicated by a decrease in availability or production of the targeted protein: a level of the targeted protein during or after treatment that is significantly lower than the level before treatment, is indicative of efficacy. A level that is lower during or after treatment can be shown, for example, by decreased serum or urinary levels of targeted protein, or decreased ex vivo production of the targeted protein. A level that is “significantly lower”, as used herein, is a level that is less than the amount that is typically found in control individual(s) or control sample(s), or is less in a comparison of disease in a population associated with the other bands of measurement (e.g., the mean or median, the highest quartile or the highest quintile) compared to lower bands of measurement (e.g., the mean or median, the other quartiles; the other quintiles).

For example, the level of the targeted protein (e.g., in a blood or serum sample, or in a tissue sample) is assessed in a sample from an individual before treatment with an therapeutic targeting agent or other therapeutic agent; and during or after treatment with the therapeutic targeting agent or other therapeutic agent, and the levels are compared. A level of the targeted protein during or after treatment that is significantly lower than the level of the targeted protein before treatment, is indicative of efficacy of treatment with the therapeutic targeting agent or other therapeutic agent. In another aspect, production of the targeted protein is analyzed in a first test sample from the individual, and is also determined in a second test sample from the individual, during or after treatment, and the level of production in the first test sample is compared with the level of production in the second test sample. A level in the second test sample that is significantly lower than the level in the first test sample is indicative of efficacy of treatment.

In another embodiment, in vivo methods as described above can be used to compare images before and after treatment with a therapeutic targeting agent or other therapeutic agent. The extent, size, location and/or number of neoplasms or of angiogenesis or neovasculature in vivo before treatment is compared with the extent, size, location and/or number during or after treatment. The efficacy of treatment is indicated by a decrease the extent, size, location and/or number of neoplasms, or a decrease in the extent of new blood vessel growth (angiogenesis or neovasculature), as indicated by decreased concentrations of imaging agents. Alternatively, the ex vivo methods as described above can be used to compare biopsy samples before and after treatment with a therapeutic targeting agent or other therapeutic agent. The extent, size, location and/or number of neoplasms or of angiogenesis or neovasculature in a sample before treatment is compared with the extent, size, location and/or number in a sample during or after treatment. The efficacy of treatment is indicated by a decrease the extent, size, location and/or number of neoplasms, or the size, location(s) of new blood vessel growth, as indicated by decreased concentrations of imaging agents. In another embodiment, in vivo methods as described above can be used to image before, during and after treatment with a therapeutic targeting agent or other therapeutic agent. For example, the extent, size, location and/or number of neoplasms or of angiogenesis or neovasculature can be assessed by in vivo imaging, and a therapeutic agent is then administered to the individual. Continued, continuous or subsequent imaging of the individual can reveal real-time targeting and destruction of neoplasm cells or of new blood vessel growth (angiogenesis or neovasculature).

In another embodiment of the invention, the level of the targeted protein can be used to assess a sample for the presence of aggressive disease and/or to assess prognosis for the patient from whom the tissue sample was obtained. Because the presence of the targeted protein is indicative of neoplasm, the amount of the targeted protein is indicative of the degree of aggression of disease: higher amounts of the targeted protein are indicative of greater extent of disease, which similarly corresponds to a poorer prognosis. Aggressive disease will show an increased amount of the targeted protein in neoplasms, compared to less aggressive disease. For example, in one aspect of the invention, an individual can be assessed to determine the targeted protein level in different tissues, cells and/or body fluids. Blood, serum, plasma or urinary levels of the targeted protein, or ex vivo production of the targeted protein, can be assessed. A level of the targeted protein that is significantly higher is indicative or aggressive disease and/or poorer prognosis. A level that is “significantly higher”, as used herein, is a level that is greater than the amount that is typically found in a control individual(s) or control sample(s), or is greater in a comparison of disease in a population associated with the other bands of measurement (e.g., the mean or median, the highest quartile or the highest quintile) compared to lower bands of measurement (e.g., the mean or median, the other quartiles; the other quintiles).

These embodiments above can similarly be used for assessment of treatment to enhance angiogenesis or development of neovasculature. The methods are performed as described, except that in these embodiments, efficacy is indicated by increased level of angiogenesis or of neovasculature as indicated by increased concentrations of imaging agents.

Tissue Engineering

Because certain proteins have been identified as being prevalent on tumor endothelium, as described herein, methods are now available to create cell types in culture that are more similar to those in vivo. (See, e.g., Engelmann, K. Et al., Exp Ehye Res (2004) 78(3):573-8; Kirkpatrick, C. J. et al., biomol. Eng. (2002):19(2-6):211-7; Nugent, H. M. and Edelman, E. R., Circ. Res. (2003) 92(10):1068-780). Tumor cells in vitro that are more similar to those in vivo, by virtue of producing similar panels of proteins on the endothelial surface, provide a better tool for assessing agents that may be useful in therapies such as the therapies described herein. Cells can be modified, for example, by incorporation of nucleic acids or vectors expressing proteins that are produced in excess in neoplasms, compared to expression in normal cells. Such modified cells allow more accurate assessment of effects of a potential therapeutic agent on neoplasm cells.

Antibodies of the Invention

In another aspect, the invention provides antibodies to certain targeted proteins, that can be used, for example, in the methods of the invention. The term, “antibody,” is described above. The invention provides polyclonal and monoclonal antibodies that bind to a targeted protein. The term “monoclonal antibody” or “monoclonal antibody composition”, as used herein, refers to a population of antibody molecules that contain only one species of an antigen binding site capable of immunoreacting with a particular epitope of the targeted protein. Polyclonal antibodies can be prepared as described above by immunizing a suitable subject with a desired immunogen, e.g., the targeted protein or a fragment or derivative thereof. The antibody titer in the immunized subject can be monitored over time by standard techniques, such as with an enzyme linked immunosorbent assay (ELISA) using immobilized polypeptide. If desired, the antibody molecules directed against the targeted protein can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein (1975) Nature, 256:495-497, the human B cell hybridoma technique (Kozbor et al. (1983) Immunol. Today, 4:72), the EBV-hybridoma technique (Cole et al. (1985), Monoclonal Antibodies and Cancer Therapy, Alan R. Liss, Inc., pp. 77-96) or trioma techniques. The technology for producing hybridomas is well known (see generally Current Protocols in Immunology (1994) Coligan et al. (eds.) John Wiley & Sons, Inc., New York, N.Y.). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cell lines can be applied for the purpose of generating a monoclonal antibody to a targeted protein (see, e.g., Current Protocols in Immunology, supra; Galfre et al. (1977) Nature, 266:55052; R. H. Kenneth, in Monoclonal Antibodies: A New Dimension In Biological Analyses, Plenum Publishing Corp., New York, N.Y. (1980); and Lerner (1981) Yale J. Biol. Med., 54:387-402. Moreover, the ordinarily skilled worker will appreciate that there are many variations of such methods that also would be useful.

Alternative to preparing monoclonal antibody-secreting hybridomas, a monoclonal antibody to a targeted protein can be identified and isolated by screening a recombinant combinatorial immunoglobulin library (e.g., an antibody phage display library) with the targeted protein, to thereby isolate immunoglobulin library members that bind to the targeted protein. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology, 9:1370-1372; Hay et al. (1992) Hum. Antibod. Hybridomas, 3:81-85; Huse et al. (1989) Science, 246:1275-1281; Griffiths et al. (1993) EMBO J., 12:725-734.

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

In general, antibodies of the invention (e.g., a monoclonal antibody) can be used in the methods of the invention. For example, an antibody specific for a targeted protein can be used in the methods of the invention to image a neoplasm, in order to evaluate the abundance and location of the neoplasm. Antibodies can thus be used diagnostically to, for example, determine the efficacy of a given treatment regimen, by imaging before and after the treatment regimen.

The invention is further illustrated by the following Exemplification, which is not intended to be limiting in any way. The teachings of all references cited herein are incorporated by reference in their entirety.

Exemplification: Subtractive Proteomic Mapping of the Endothelial Surface in Lung and Solid Tumors for Tissue-Specific Therapy

Materials. Antibodies were obtained: AnnA1, AnnA8, EphA5, Eph A7, ACE, APN, caveolin-1, E-cadherin, Tie 2, and VE-Cadherin from Santa Cruz Biotechnology (Santa Cruz, Calif.); aquaporin-1, was obtained form BD Biosciences/Pharmingen (San Diego, Calif.); β-COP, beta-actin, MΦ, and fibroblast surface protein from Sigma (Saint Louis, Mo.); RAGE from Affinity Bioreagents (Golden, Colo.); TfnR, VEGF R2, and ECE were from Zymed Lab, Inc. (San Francisco, Calif.); nucleolin from Leinco Technologies (St. Louis, Mo.); CD4 from Serotech (Raleigh, N.C.); DPPIV form BD Biosciences (San Diego, Calif.); TM from Covance (Princeton, N.J.); MPO from Accurate Chemicals (Westbury, N.Y.), and Vitamin D binding protein from DAKO (Carpinteria, Calif.). Antibodies against carbonic anhydrase IV were a kind gift of W. S. Sly, St. Louis University (St. Louis, Mo.); APP, PV-1, and podocalyxin were produced in house; seven transmembrane receptor was a kind gift of Dr. Shigehisa Hirose, Tokyo Institute of Technology (Yokohama, Japan); OX-45 was a kind gift of Dr. Neil Barclay, University of Oxford (Oxford, UK); galectin 1 was a kind gift of Dr. M. Huflejt, Sidney Kimmel Cancer Center (San Diego, Calif.); VEGF receptor 1 was a kind gift of Dr. D. Sanger, Beth Israel Deaconess Medical Center (Boston Mass.); endoglin was a kind gift of Dr. Yamashita, Ludwig Institute for Cancer Research; neurophilin 1 was a kind gift of Dr. Ginty, Johns Hopkins School of Medicine (Baltimore, Md.); C-CAM1 was a kind gift of Sue-Hwa Lin, The University of Texas M.D. Anderson Cancer Center (Houston, Tex.).

Expression profiling in vivo of candidate proteins. To assess expression of candidate endothelial cell proteins in different tissues, proteins from the tissue homogenate and β lated from normal rat lung, heart, kidney, liver, and/or tumor-bearing lungs were solubilized with CLB buffer (6M urea, 0.5M Tris pH 6.8, 9 mM EDTA, 9% sodium dodecyl sulfate), separated by SDS-PAGE, and electrotransferred to nitrocellulose filters for immunoblotting with the appropriate antibodies as described (Schnitzer, J. E., McIntosh, D. P., Dvorak, A. M., Liu, J. & Oh, P. Separation of caveolae from associated microdomains of GPI-anchored proteins. Science 269, 1435-9 (1995)).

Rat tumor models. Female Fisher rats (100-150 gms) were injected via the tail vein with a cell suspension of 13762 breast adenocarcinoma cells to give ample, well circumscribed, and highly vascularized tumors in the lung. To create a maximum density of tumor lesions of 3-8 mm in diameter that are clearly visible in the lungs, we injected 5×10⁵ 13762 cells 14-15 days prior to perfusion and isolation of tumor-bearing lung P. To obtain a few well-circumscribed tumors of 3-6 mm in diameter, we injected 1×10⁵ cells 21 days prior to performing the imaging experiments.

Gamma scintigraphic imaging and biodistribution analysis. Monoclonal antibodies were isolated using GammaBind Plus Sepharose (Amersham, Piscataway, N.J.) and conjugated to ¹²⁵I using Iodogen as described (McIntosh, D. P., Tan, X.-Y., Oh, P. & Schnitzer, J. E. Targeting endothelium and its dynamic caveolae for tissue-specific transcytosis in vivo: A pathway to overcome cell barriers to drug and gene delivery. Proc. Natl. Acad. Sci. USA 99, 1996-2001 (2002)). Biodistribution analysis was performed as described (id). Imaging was performed using an A-SPECT imaging system, a dedicated small animal radiotracer imaging system (Gamma Medica, Inc., Northridge, Calif.) (McElroy, D. et al. Performance evaluation of A-SPECT: A high resolution desktop pinhole SPECT system for imaging small animals. IEEE Trans Nucl Sci NS 49, 2139-2147 (2002)), fitted with a parallel-hole collimator. Normal and tumor-bearing female Fisher rats were anaesthetized and injected via the tail vein with ¹²⁵I-labeled monoclonal antibody (5 ug IgG; 10 uCi/ug) before being subjected to planar gamma scintigraphic imaging captured over 10 min. For tomographic studies, the images were captured in 6C increments for 64 frames (30 sec/frame). After whole body imaging, in some cases, the lungs were excised for planar imaging captured over 10 min ex vivo.

Proteomic analysis. We used a three-pronged approach to resolve proteins/peptides for MS analysis: i) resolving the proteins of P and V from various normal organs or tumors by high resolution 2D gel analysis before excising apparently organ or tumor-specific protein spots for MS analysis; ii) separating proteins in P and V on 1D SDS-PAGE gels, cutting specific bands of interest or the whole gel lane into 50 slices, and analysing the proteins in each of the gel slices by MS; and iii) using Multi-dimensional Protein Identification Technology (MudPIT) to analyse tryptic peptides from a complex mixture of proteins extracted directly from the whole membrane P and V isolates. Each gel spot/slice was de-stained and digested overnight with trypsin before extracting the cleaved peptides from the gel and then loading them onto a reverse phase C-18 micro-column for gradient acetonitrile elution directly into the mass spectrometer (LCQ Deca XP ion trap mass spectrometer (ThermoFinnigan, San Jose, Calif.) equipped with a modified micro-electrospray ionization source from Mass Evolution (Spring, Tex.)). For MudPIT, 150 μg of complex peptide mixture was separated by 2-D liquid chromatography a micro-column packed with three phases of chromatographic material as follows: 8.5 cm of 5 μm C₁₈ reversed phase material (Polaris C18-A, Metachem, Torrance, Calif.), then 4 cm of 5 μm, 300 Å strong cation exchanger (PolyLC, Columbia, Md.) and lastly 3.5 cm of C₁₈ material using a helium pressure cell operated at 600-900 psi (Mass Evolution, Spring, Tex.). Peptides were directly eluted into the mass spectrometer using a 2D chromatography with 18 step-elutions from the strong cation exchanger followed by a gradient elution of the reversed phase material. Operation of the quarternary Agilent 1100 HPLC pump and the mass spectrometer was fully automated during the entire procedure using the Excalibur 1.2 data system (ThermoFinnigan, San Jose, Calif.). Continuous cycles of one full scan (m/z 400 to 1400) followed by 3 data-dependent MS/MS measurements at 35% normalized collision energy were performed. MS/MS measurements were allowed for the 3 most intense precursor ions with an enabled exclusion list of 25 m/z values (+/−1.5 Da) or a maximum time limit of 5 minutes. The zoom scan function to determine the charge state was disabled in order to increase the duty cycle of the instrument.

Database search and in silico analysis of tandem mass spectra. MS/MS spectra were extracted from raw files requiring a minimum of 21 signals with an intensity of at least 4.75×10⁴ a.u. Extracted MS/MS spectra were automatically assigned to the best matching peptide sequence using the SEQUEST algorithm and the Sequest Browser software package (ThermoFinnigan, San Jose, Calif.). SEQUEST searches were performed using a rat protein database containing 40,800 protein sequences downloaded as FASTA formated sequences from ENTREZ (NCBI; http://www.ncbi.nlm.nih.gov/Entrez). Sequence redundancies were removed using Perl script. The peptide mass search tolerance was set to 3 Da. Spectral matches were retained with a minimal cross-correlation score (XCorr) of 1.5, 2.2 and 3.3 for charge states +1, +2 and +3 respectively. DeltaCN (top match's XCorr minus the second-best match's XCorr devided by top match's XCorr) had to be equal or less than 0.07. Retained spectral matches were filtered and re-assigned to proteins using DTASelect. DTASelect outputs of independent measurements were entered into Accessible Vascular Targets database (AVATAR). AVATAR was designed to store a large amount of mass spectrometric data and to provide tools to analyze the data to extact valuable information. We used relational models for database design based on Entity-Relationship and implemented the database in the MySQL relational database management system (MySQL Inc., Seattle Wash.) to support database query and management. This relational database plus Perl-based user-friendly interface have greatly improved data organization, data consistency and integrity, and facilitated data comparison and information retrieval. In the case of the 1D gel and MudPIT approaches, AVATAR is used to subtract the data to find proteins detected on the tumor but not normal endothelium.

In silico bioinformatic interrogation. To identify possible candidates for intravenously-accessible targets from the subset of proteins identified as lung or tumor induced, we determined their currently known membrane-associated structure (bilayer spanning vs. lipid anchor (intra- or extracellular) vs. peripheral interaction) as per scientific reports and/or protein databases, such as SwissProt (http://us.expasy.org/sprot/sprot-top.html) 52- and the National Center Biotechnology Information (NCBI; http://www.ncbi.nlm.nih.gov/entrez/query.fcgi) protein database. We also used web-based prediction programs to identify candidates that may harbor transmembrane spanning alpha helices (TMpred—Prediction of Transmembrane Regions and Orientation; http://www.ch.embnet.org/software/TMPRED_form.html) or glycosylation sites (indicating a possible ectodomain exposed to the circulating blood; Prosite Scan, http://npsa-pbil.ibcp.fr/cgi-bin/npsa_automat.pl?page=npsa_prosite.html). Only 100% probabilities were taken into consideration.

Results

To begin to investigate endothelial cell surface heterogeneity in vivo, we performed subcellular fractionation to isolate luminal endothelial cell plasma membranes (P) and caveolae (V) directly from normal organs (Oh, P. & Schnitzer, J. E. in Cell Biology: A Laboratory Handbook (ed. Celis, J.) 34-36 (Academic Press, Orlando, 1998); Schnitzer, J. E., McIntosh, D. P., Dvorak, A. M., Liu, J. & Oh, P. Separation of caveolae from associated microdomains of GPI-anchored proteins. Science 269, 1435-9 (1995)). P and V displayed _{C}20-fold enrichment for endothelial cell surface and caveolar markers (angiotensin converting enzyme (ACE), VE-cadherin, and caveolin-1) whereas proteins of intracellular organelles (e.g. _{C}-COP for Golgi), other tissue cells (e.g. E-cadherin for epithelium, fibroblast surface protein), and blood (e.g. glycophorin A, CD4, CD11) were _{C}20-fold depleted (data not shown; data in accordance with previous results (Schnitzer, J. E. in Vascular Endothelium: Physiology, pathology and therapeutic opportunities. (eds. Born, G. V. R. & Schwartz, C. J.) 77-95 (Schattauer, Stuttgart, 1997); Oh, P. & Schnitzer, J. E. in Cell Biology: A Laboratory Handbook (ed. Celis, J.) 34-36 (Academic Press, Orlando, 1998); Schnitzer, J. E., McIntosh, D. P., Dvorak, A. M., Liu, J. & Oh, P. Separation of caveolae from associated microdomains of GPI-anchored proteins. Science 269, 1435-9 (1995)). This quality control was applied to each isolate.

We analysed P by 2-D gel electrophoresis to produce high-resolution vascular endothelial protein maps of the major rat organs that were distinct and much reduced in complexity from that of the starting tissue homogenate (data not shown). Differential spot analysis revealed many distinct proteins in P vs. the homogenate and in P between organs, and Western analysis confirmed this heterogeneity further. In many cases, antigens difficult to detect in tissue homogenates were readily apparent in P, reflecting the significant enrichment and increased sensitivity provided by subfractionation to unmask proteins located on the endothelial cell surface. Unique “molecular fingerprints or signatures” that may include tissue- and cell-specific proteins were apparent for each endothelia.

Subtractive Analysis and Profiling.

Tumor-Induced Endothelial Cell Proteins.

To determine whether the tumor microenvironment in the lung is sufficiently different to induce new endothelial protein expression, we isolated P and V from normal rat lungs and lungs bearing breast adenocarcinomas. As above, these isolates were significantly enriched relative to the tissue homogenates in endothelial cell surface markers (caveolin, 5′nucleotidase, ACE, and VE-cadherin) while being markedly depleted in markers of possible contaminants, including β-COP, CD4, CD11, glycophorin A, fibroblast surface protein and galectin-1 (which is expressed by the tumor cells (Perillo, N. L., Marcus, M. E. & Baum, L. G. Galectins: versatile modulators of cell adhesion, cell proliferation, and cell death. J Mol Med 76, 402-12. (1998))) (data not shown). Tumor P was also enriched in angiogenesis markers relative to normal lung P. Lastly, markers of immune cells known to infiltrate solid tumors were detected in tumor homogenates but not tumor P.

We used 2-D gels to visualize several hundred protein spots in lung P vs. tumor lung P. These maps were reproducible. Multiple protein spots were detected in tumor P but not normal P. Prominent 2-D spots easily detected in tumor P were not detected in the homogenates, consistent with the small percentage of endothelial cell plasma membranes in the tumors. Tissue subfractionation appeared necessary to unmask differentially expressed tumor vascular proteins obscured by the molecular complexity of the total tumor.

We again applied a subtractive proteomic approach using antibody and MS analysis of P and V to identify so far 15 differentially expressed proteins, including proteins already implicated in tumor angiogenesis: VEGF receptors-1 and -2, Tie2, aminopeptidase-N, endoglin, C-CAM-1, and neuropilin-1 (Ferrara, N. VEGF and the quest for tumor angiogenesis factors. Nat Rev Cancer 2, 795-803 (2002); Kerbel, R. & Folkman, J. Clinical translation of angiogenesis inhibitors. Nat Rev Cancer 2, 727-39 (2002)). These proteins were enriched in tumor P relative to tumor homogenates, consistent with proper subfractionation. Eight new tumor-induced vascular proteins were also identified: AnnexinA1, AnnexinA8, EphrinA5, EphrinA7, myeloperoxidase, nucleolin, transferrin receptor, and vitamin D-binding protein. Consistent with the subtractive screen hypothesis, 12 of 15 proteins were much more evident in tumor P than normal P.

Expression profiling using P from major organs revealed almost all of these proteins exist at the cell surface of at least one major organ albeit mostly at levels much less than the tumor endothelial cell surface. One promising tumor candidate target was the 34 kDa protein recognized by AnnexinA1 (AnnA1) antibodies only in tumor P. Tissue immunohistochemistry confirmed tumor blood vessel reactivity (data not shown). Thus, the tumor microenvironment appeared to induce distinct protein expression on the endothelial cell surface. AnnexinA1 and its use in tumor imaging and treatment is described in greater detail in Attorney Docket No. 3649.1000-000, filed on Jun. 2, 2004, entitled, “VASCULAR TARGETS FOR DETECTING, IMAGING AND TREATING PRIMARY AND METASTATIC TUMORS”, Attorney Docket no. 3649.1000-003, filed on even date herewith, entitled, “VASCULAR TARGETS FOR DETECTING, IMAGING AND TREATING NEOPLASIA OR NEOVASCULATURE” the teachings of which are incorporated herein by reference in their entirety.

Targeting and Imaging of Solid Tumors.

Annexins are cytosolic proteins that can associate with cell membranes in a calcium-dependent manner (Gerke, V. & Moss, S. E. Annexins: from structure to function. Physiol Rev 82, 331-71 (2002)). Some annexins may translocate the lipid bilayer to the external cell surface (id). To test whether AnnA1 is sufficiently exposed and tumor vessel-specific to permit immunotargeting in vivo, we performed whole body imaging using ¹²⁵I-labeled AnnA1 monoclonal antibodies. gamma-scintigraphic planar images captured 4 hours postinjection showed a distinct focus of radioactivity in the lung and little signal elsewhere in the body. Non-targeting ¹²⁵I-labeled IgGs did not target (data not shown). When the lungs were imaged ex vivo, we observed ¹²⁵I-AnnA1 antibody accumulation in the tumor as a hot spot corresponding to visible tumors. Targeting was prevented by 30-fold excess unlabeled AnnA1 IgG but not control IgG (data not shown). Region of interest and biodistribution analysis confirmed targeting in vivo with an average tumor accumulation at 2 hours of 34% ID/g, which compared favourably to VEGF receptor antibodies which accumulated at 6.4% ID/g of tumor. When injected into rats without tumors, ¹²⁵I-AnnA1 antibodies showed no targeting of normal organs, including lung at levels <1% ID/g of tissue, whereas VEGF receptor antibody accumulation was greater in multiple organs. The uptake ratio in rat tumor-bearing lungs vs. normal lungs was up to 2.0 and 70 for antibodies to VEGF receptor and AnnA1, respectively. Thus, gamma-scintigraphic imaging rapidly validated AnnA1 as a tumor target that is readily accessible to antibody injected intravenously for tumor targeting and imaging in vivo. AnnA1 appeared to be selectively externalised on the endothelial cell surface by the solid tumors.

Radio-Immunotherapy of Solid Tumors.

Because many tumor-bearing rats imaged with the ¹²⁵I-AnnA1 antibody survived, we performed a survival study and recorded animal body weights. 80% of the animals survived 8 days or longer after treatment with ¹²⁵I-AnnA1 antibody. The ¹²⁵I-IgG-treated and untreated rats all died within 7 days. The body weights of all tumor-bearing rats began to drop 7-10 days after tumor cell inoculation. The control rats continued to decrease in body weight to 23-30% less than normal at their death. In contrast, rats treated with ¹²⁵I-AnnA1 IgG began to gain weight within 3-4 days and reached a normal body weight after 25 days. This increased survival was striking because in this model many animals die within 2-4 days of treatment and thus may lack sufficient time to benefit from treatment. The survival rate of rats surviving the first week approached 90%. The one rat that died after two weeks required euthanasia because of a leg tumor and large tail tumor that were not apparent when treated. Thus, a single injection of ¹²⁵I-AnnA1 antibody caused significant remission even in advanced disease.

AnnA1 in Human Tumor Neovasculature.

We immunostained tissue sections of human solid tumors. AnnA1 antibody labelled blood vessels of human prostate, liver, kidney, and lung tumors but not matched normal tissues. Antibodies to PECAM stained both normal and tumor blood vessels. The lack of AnnA1 expression in vascular endothelium of multiple normal organs has been reported previously (Dreier, R., Schmid, K. W., Gerke, V. & Riehemann, K. Differential expression of annexins I, II and IV in human tissues: an immunohistochemical study. Histochem Cell Biol 110, 137-48 (1998); Eberhard, D. A., Brown, M. D. & VandenBerg, S. R. Alterations of annexin expression in pathological neuronal and glial reactions. Immunohistochemical localization of annexins I, II (p36 and p11 subunits), IV, and VI in the human hippocampus. Am J Pathol 145, 640-9 (1994); Ahn, S. H., Sawada, H., Ro, J. Y. & Nicolson, G. L. Differential expression of annexin I in human mammary ductal epithelial cells in normal and benign and malignant breast tissues. Clin Exp Metastasis 15, 151-6 (1997); McKanna, J. A. & Zhang, M. Z. Immunohistochemical localization of lipocortin 1 in rat brain is sensitive to pH, freezing, and dehydration. J Histochem Cytochem 45, 527-38 (1997)). Thus, AnnA1 was selectively detected on the neovascular endothelium of multiple human solid tumors.

Discussion

Because of poor access inside many tissues, antibodies injected intravenously usually require significantly higher doses (250 ug/kg (Bredow, S., Lewin, M., Hofmann, B., Marecos, E. & Weissleder, R. Imaging of tumor neovasculature by targeting the TGF-beta binding receptor endoglin. Eur J Cancer 36, 675-81 (2000)) compared to 20 ug/kg used here) to get a small percentage into the tissue that binds and can then be visualized clearly a day or so later once the blood is cleared of unwanted, interfering background signal. As described herein, however, binding is direct and unhindered by barriers so that both tissue accumulation and blood clearance are rapid, thereby providing striking images within minutes to hours. The rapidity and high levels of specific targeting observed here meet the theoretical expectation of the vascular targeting strategy (Schnitzer, J. E. Vascular targeting as a strategy for cancer therapy. N Engl J Med 339, 472-4 (1998)). Although a fast emerging standard for assessing targeting and specificity (Massoud, T. F. & Gambhir, S. S. Molecular imaging in living subjects: seeing fundamental biological processes in a new light. Genes Dev 17, 545-80 (2003); Herschman, H. R. Molecular imaging: looking at problems, seeing solutions. Science 302, 605-8 (2003); Rudin, M. & Weissleder, R. Molecular imaging in drug discovery and development. Nat Rev Drug Discov 2, 123-31 (2003); Weissleder, R. Scaling down imaging: molecular mapping of cancer in mice. Nat Rev Cancer 2, 11-8 (2002)), whole body imaging may be underutilized in target validation despite being non-invasive and highly sensitive.

The neoplasm-specific endothelial cell surface proteins described herein, when used as vascular targets, allow targeting of neoplasms in vivo. We expect that site-directed vascular and caveolar targeting will benefit both drug and gene delivery in the treatment of many diseases (Carver, L. A. & Schnitzer, J. E. Caveolae: mining little caves for new cancer targets. Nat Rev Cancer 3, 571-81 (2003)). We demonstrate here using an experimental rat tumor model that monoclonal antibodies to AnnA1 can effectively direct low levels of radionuclides (100_{C}Ci) to concentrate in, and thus destroy, solid tumors and ultimately increase animal survival. Because AnnA1 is also selectively detected in multiple human solid tumors, this target may similarly help image and treat human disease.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of delivering an agent to, into and/or across vascular endothelium in a neoplasm-specific manner, comprising contacting luminal surface and/or caveolae of vasculature with an agent that specifically binds a targeted protein expressed on endothelial cell surface.

2. The method of claim 1, wherein the targeted protein is selected from the group consisting of: AnnA1, AnnA8, EphA5, EphA7, myeloperoxidase, nucleolin, transferrin receptor, and vitamin D binding receptor.

3. A method of treating neoplasia in an individual, comprising administering to the individual a therapeutic targeting agent that specifically binds a targeted protein expressed on endothelial cell surface.

4. The method of claim 3, wherein the therapeutic targeting agent is an antibody to the targeted protein.

5. The method of claim 3, wherein the therapeutic targeting agent is a specific binding partner of the targeted protein.

6. The method of claim 3, wherein the therapeutic targeting agent is an agent that comprises an active agent component and a targeting agent component, wherein the active agent component is selected from the group consisting of: a radionuclide; a chemotherapeutic agent; an immune stimulatory agent; an anti-neoplastic agent: an anti-inflammatory agent; a pro-inflammatory agent; a pro-apoptotic agent; a pro-coagulant; a toxin; an antibiotic; a hormone; an enzyme; a protein (e.g., a recombinant protein or a recombinant modified protein) a carrier protein (e.g., albumin, modified albumin); a lytic agent; a small molecule; aptamers; cells, including modified cells; vaccine-induced or other immune cells; nanoparticles (e.g., albumin-based nanoparticles); transferrins; immunoglobulins; multivalent antibodies; lipids; lipoproteins; liposomes; an altered natural ligand; a gene or nucleic acid; RNA; siRNA; a viral or non-viral gene delivery vector; a prodrug; or a promolecule.

7. The method of claim 3, wherein the targeted protein is selected from the group consisting of: AnnA1, AnnA8, EphA5, EphA7, myeloperoxidase, nucleolin, transferrin receptor, and vitamin D binding receptor.

8. The method of claim 3, wherein the targeted protein is selected from the group consisting of: VEGF receptor 1, VEGF receptor 2, Tie-2, aminopeptidase N, endoglin, C-CAM-1, and neuropilin-1; and the targeting agent is used for treatment of a neoplasm.

9. A physiological composition comprising a therapeutic targeting agent.

10. A method of performing physical imaging of an individual, comprising administering to the individual an imaging agent comprising a targeting agent component and an imaging agent component, wherein the targeting agent component specifically binds to a targeted protein expressed on the endothelial cell surface.

11. The method of claim 11, wherein the targeted protein is selected from the group consisting of: AnnA1, AnnA8, EphA5, EphA7, myeloperoxidase, nucleolin, transferrin receptor, and vitamin D binding receptor.

12. The method of claim 11, wherein the targeted protein is selected from the group consisting of: VEGF receptor 1, VEGF receptor 2, Tie-2, aminopeptidase N, endoglin, C-CAM-1, and neuropilin-1; and the imaging agent is for imaging of a neoplasm

13. The method of claim 11, wherein the imaging agent component is selected from the group consisting of: a radioactive agent (e.g., radioiodine (125I, 131I); technetium; yttrium; 35S or 3H) or other radioisotope or radiopharmaceutical; a contrast agent (e.g., gadolinium; manganese; barium sulfate; an iodinated or noniodinated agent; an ionic agent or nonionic agent); a magnetic agent or a paramagnetic agent (e.g., gadolinium, iron-oxide chelate); liposomes (e.g., carrying radioactive agents, contrast agents, or other imaging agents); nanoparticles; ultrasound agents (e.g., microbubble-releasing agents); a gene vector or virus inducing a detecting agent (e.g., including luciferase or other fluorescent polypeptide); an enzyme (horseradish peroxidase, alkaline phosphatase, β-galactosidase, or acetylcholinesterase); a prosthetic group (e.g., streptavidin/biotin and avidin/biotin); a fluorescent material (e.g., umbelliferone, fluorescein, fluorescein isothiocyanate, rhodamine, dichlorotriazinylamine fluorescein, dansyl chloride or phycoerythrin); a luminescent material (e.g., luminol); and a bioluminescent material (e.g., luciferase, luciferin, aequorin).

14. A method of assessing an individual for the presence or absence of neoplasia, comprising:
a) administering to the individual an imaging agent that comprises an imaging agent component and a targeting agent component, wherein the targeting agent component specifically binds to a targeted protein expressed on vascular endothelium, and
b) assessing the individual for the presence or absence of a concentration of the imaging agent,
wherein the presence of a concentration of the imaging agent is indicative of the presence of neoplasia.

15. The method of claim 14, wherein the targeting agent component is an antibody to the targeted protein.

16. The method of claim 14, wherein the targeting agent component is a specific binding partner of the targeted protein.

17. A method of assessing an individual for the presence or absence of neoplasia, comprising:
a) administering to the individual an agent of interest that comprises an imaging agent component and a targeting agent component, wherein the targeting agent component specifically binds to a targeted protein expressed on vascular endothelium;
b) taking a biopsy sample from the individual;
c) assessing the biopsy sample for the presence or absence of a concentration of the agent of interest,
wherein the presence of a concentration of the agent of interest is indicative of the presence of neoplasia.

18. A method of delivering an imaging agent in a neoplasm-specific manner, comprising contacting luminal surface and/or caveolae of vasculature with an imaging agent that comprises an imaging agent component and a targeting agent component, wherein the targeting agent component specifically binds to a targeted protein expressed on an endothelial cell surface of the neoplasm.

19. The method of claim 18, wherein the targeted protein is selected from the group consisting of: AnnA1, AnnA8, EphA5, EphA7, myeloperoxidase, nucleolin, transferrin receptor, and vitamin D binding receptor.

20. The method of claim 18, wherein the targeted protein is selected from the group consisting of: VEGF receptor 1, VEGF receptor 2, Tie-2, aminopeptidase N, endoglin, C-CAM-1, and neuropilin-1.

21. The method of claim 18, wherein the targeted protein is AnnA1 or vitamin D binding protein.

22. The method of claim 18, wherein the imaging agent component is selected from the group consisting of: a radioactive agent (e.g., radioiodine (125I, 131I); technetium; yttrium; 35S or 3H) or other radioisotope or radiopharmaceutical; a contrast agent (e.g., gadolinium; manganese; barium sulfate; an iodinated or noniodinated agent; an ionic agent or nonionic agent); a magnetic agent or a paramagnetic agent (e.g., gadolinium, iron-oxide chelate); liposomes (e.g., carrying radioactive agents, contrast agents, or other imaging agents); nanoparticles; ultrasound agents (e.g., microbubble-releasing agents); a gene vector or virus inducing a detecting agent (e.g., including luciferase or other fluorescent polypeptide); an enzyme (horseradish peroxidase, alkaline phosphatase, β-galactosidase, or acetylcholinesterase); a prosthetic group (e.g., streptavidin/biotin and avidin/biotin); a fluorescent material (e.g., umbelliferone, fluorescein, fluorescein isothiocyanate, rhodamine, dichlorotriazinylamine fluorescein, dansyl chloride or phycoerythrin); a luminescent material (e.g., luminol); and a bioluminescent material (e.g., luciferase, luciferin, aequorin).

23. A method of delivering an imaging agent in a neoplasm-specific manner to a tissue sample, comprising contacting the tissue sample with an imaging agent that comprises an imaging agent component and a targeting agent component, wherein the targeting agent component specifically binds to a targeted protein expressed on an endothelial cell surface of the neoplasm.

24. A method of assessing response of an individual to treatment with an therapeutic targeting agent, wherein the therapeutic targeting agent specifically binds a targeted protein expressed on endothelial cell surface, comprising:
a) assessing the level of the targeted protein in a sample from the individual before treatment with the therapeutic targeting agent;
b) assessing the level of the targeted protein in a sample from the individual during or after treatment with the therapeutic targeting agent;
c) comparing the level before treatment with the level during or after treatment,
wherein a level of the targeted during or after treatment that is significantly lower than the level of the targeted protein before treatment, is indicative of efficacy of treatment with the therapeutic targeting agent.

25. A method of delivering an agent to, into and/or vascular endothelium in an neovasculature-specific manner, comprising contacting luminal surface and/or caveolae of vasculature with an agent that specifically binds a targeted protein expressed on endothelial cell surface.

26. The method of claim 25, wherein the targeted protein is selected from the group consisting of: AnnA1, AnnA8, EphA5, EphA7, myeloperoxidase, nucleolin, transferrin receptor, and vitamin D binding receptor.

27. A method of treating angiogenesis or neovasculature in an individual, comprising administering to the individual a therapeutic targeting agent that specifically binds a targeted protein expressed on endothelial cell surface.

28. The method of claim 27, wherein the therapeutic targeting agent is an antibody to the targeted protein.

29. The method of claim 27, wherein the therapeutic targeting agent is a specific binding partner of the targeted protein.

30. The method of claim 27, wherein the therapeutic targeting agent is an agent that comprises an active agent component and a targeting agent component, wherein the active agent component is selected from the group consisting of a radionuclide; a chemotherapeutic agent; an immune stimulatory agent; an anti-neoplastic agent: an anti-inflammatory agent; a pro-inflammatory agent; a pro-apoptotic agent; a pro-coagulant; a toxin; an antibiotic; a hormone; an enzyme; a protein (e.g., a recombinant protein or a recombinant modified protein) a carrier protein (e.g., albumin, modified albumin); a lytic agent; a small molecule; aptamers; cells, including modified cells; vaccine-induced or other immune cells; nanoparticles (e.g., albumin-based nanoparticles); transferrins; immunoglobulins; multivalent antibodies; lipids; lipoproteins; liposomes; an altered natural ligand; a gene or nucleic acid; RNA; siRNA; a viral or non-viral gene delivery vector; a prodrug; and a promolecule, and wherein the targeting agent component specifically binds to a targeted protein.

31. The method of claim 27, wherein the targeted protein is selected from the group consisting of: AnnA1, AnnA8, EphA5, EphA7, myeloperoxidase, nucleolin, transferrin receptor, and vitamin D binding receptor.

32. The method of claim 27, wherein the targeted protein is selected from the group consisting of: VEGF receptor 1, VEGF receptor 2, Tie-2, aminopeptidase N, endoglin, C-CAM-1, and neuropilin-1; and the targeting agent is used for treatment of angiogenesis or neovasculature

33. A method of assessing an individual for the presence or absence of angiogenesis or of neovasculature, comprising:
a) administering to the individual an imaging agent that comprises an imaging agent component and a targeting agent component, wherein the targeting agent component specifically binds to a targeted protein expressed on vascular endothelium, and
b) assessing the individual for the presence or absence of a concentration of the imaging agent,
wherein the presence of a concentration of the imaging agent is indicative of the presence of angiogenesis or neovasculature.

34. The method of claim 33, wherein the targeting agent component is an antibody to the targeted protein.

35. The method of claim 33, wherein the targeting agent component is a specific binding partner of the targeted protein.

36. A method of assessing an individual for the presence or absence of angiogenesis or neovasculature, comprising:
a) administering to the individual an agent of interest that comprises an imaging agent component and a targeting agent component, wherein the targeting agent component specifically binds to a targeted protein expressed on vascular endothelium;
b) taking a biopsy sample from the individual;
c) assessing the biopsy sample for the presence or absence of a concentration of the agent of interest,
wherein the presence of a concentration of the agent of interest is indicative of the presence of angiogenesis or neovasculature

37. A method of delivering an imaging agent in an neovasculature-specific manner, comprising contacting the luminal surface and/or caveolae of vasculature with an imaging agent that comprises an imaging agent component and a targeting agent component, wherein the targeting agent component specifically binds to a targeted protein expressed on an endothelial cell surface.

38. The method of claim 37, wherein the targeted protein is selected from the group consisting of: AnnA1, AnnA8, EphA5, EphA7, myeloperoxidase, nucleolin, transferrin receptor, and vitamin D binding receptor.

39. The method of claim 37, wherein the targeted protein is selected from the group consisting of: VEGF receptor 1, VEGF receptor 2, Tie-2, aminopeptidase N, endoglin, C-CAM-1, and neuropilin-1.

40. The method of claim 37, wherein the targeted protein is AnnA1 or vitamin D binding protein.

41. The method of claim 37, wherein the imaging agent component is selected from the group consisting of: a radioactive agent (e.g., radioiodine (125I, 131I); technetium; yttrium; 35S or 3H) or other radioisotope or radiopharmaceutical; a contrast agent (e.g., gadolinium; manganese; barium sulfate; an iodinated or noniodinated agent; an ionic agent or nonionic agent); a magnetic agent or a paramagnetic agent (e.g., gadolinium, iron-oxide chelate); liposomes (e.g., carrying radioactive agents, contrast agents, or other imaging agents); nanoparticles; ultrasound agents (e.g., microbubble-releasing agents); a gene vector or virus inducing a detecting agent (e.g., including luciferase or other fluorescent polypeptide); an enzyme (horseradish peroxidase, alkaline phosphatase, β-galactosidase, or acetylcholinesterase); a prosthetic group (e.g., streptavidin/biotin and avidin/biotin); a fluorescent material (e.g., umbelliferone, fluorescein, fluorescein isothiocyanate, rhodamine, dichlorotriazinylamine fluorescein, dansyl chloride or phycoerythrin); a luminescent material (e.g., luminol); and a bioluminescent material (e.g., luciferase, luciferin, aequorin).

42. A method of delivering an imaging agent in an neovasculature-specific manner to a tissue sample, comprising contacting the tissue sample with an imaging agent that comprises an imaging agent component and a targeting agent component, wherein the targeting agent component specifically binds to a targeted protein expressed on an endothelial cell surface of new blood vessel growth.

43. A method of increasing neovasculature in an individual, comprising administering to the individual an neovasculature targeting agent.

44. The method of claim 43, wherein the neovasculature targeting agent is an agent that comprises an active agent component and a targeting agent component, wherein the targeting agent component is an agent that specifically binds to a targeted protein selected from the group consisting of: AnnA1, AnnA8, EphA5, EphA7, myeloperoxidase, nucleolin, transferrin receptor, and vitamin D binding receptor.

45. A method of treating neoplasia in an individual, comprising administering to the individual a therapeutic targeting agent that comprises a targeted protein expressed on endothelial cell surface, wherein an immune response is generated against the targeted protein.

46. The method of claim 45, wherein the therapeutic targeting agent further comprises a modified cell.
